(19) United States
(12) Patent Application Publication
(10) Pub. No.: US 2015/0125879 A1
(43) Pub. Date: May 7, 2015

(54) Biodegradable Layer-by-Layer (LbL) Films for Cell Capture and Release

(71) Applicant: Massachusetts Institute of Technology, Cambridge, MA (US)

(72) Inventors: Wei Li, Lubbock, TX (US); Steven Andrew Castleberry, Boston, MA (US); Paula Therese Hammond, Newton, MA (US)

(21) Appl. No.: 14/496,588

(22) Filed: Sep. 25, 2014

Related U.S. Application Data
(60) Provisional application No. 61/882,623, filed on Sep. 25, 2013.
(60) Provisional application No. 61/883,157, filed on Sep. 26, 2013.

(51) Int. Cl. G01N 33/50; B01L 3/00

(52) U.S. Cl. CPC G01N 33/5005; B01L 3/5027; B01L 2200/10; B01L 2300/0887
USPC 435/7.23; 422/527; 435/287.1; 422/502

(57) ABSTRACT

Compositions and methods for providing devices comprised of a substrate and a Layer-By-Layer (LBL) film coated on at least a surface of the substrate, which LBL film comprises binding agents that specifically interact with cells. Such devices are useful, for example, in various cell isolation applications. Among other advantages, such devices permit isolation and release (e.g., via layer degradation) of cells under mild conditions.

### RELATED APPLICATIONS

This application claims the benefit of U.S. Provisional Application No. 61/882,623, filed on Sep. 25, 2013 and U.S. Provisional Application No. 61/883,157, filed on Sep. 26, 2013. The entire teachings of the above applications are incorporated herein by reference.

### GOVERNMENT SUPPORT

This invention was made with government support under Contract No. W911NF-07D-0004 from 13-D-0001 awarded by the Army Research Office. The government has certain rights in the invention.

### BACKGROUND OF THE INVENTION

Separation of cell types is an important capability and an active area of research in both clinical medicine and basic science. A variety of technologies and techniques have been developed to perform the task of selective isolation and separation of cells. Structures and methods for capturing and releasing cells without invasive techniques remain desirable.

### SUMMARY OF THE INVENTION

It has now been discovered that using layer-by-layer (LbL) structures and methods, it is possible to capture and release cells or other structures. Layer-By-Layer (LBL) is a robust method for creating thin films through serial assembly of individual layers that rely on complementary interactions to associate with one another. LBL films have proven to be useful in a variety of applications and contexts. The present invention encompasses the recognition that LBL films may be particularly useful in cell isolation applications (e.g., in selectively isolating cells, for example from bulk fluid). Among other things, the present invention identifies the source of various problems with certain prior art cell separation technologies, including that many such techniques place the isolated cells under excessive stresses that may, for example, reduce cell viability and/or interfere with cellular processes so that phenotype characteristics and/or other valuable information about the cells may be lost or destroyed during the isolation process.

In certain embodiments, the present invention encompasses the recognition that biodegradable LBL films, and particularly LBL films that can be degraded upon command (i.e., through the application of a particular trigger), can permit cell isolation, and/or, in some embodiments, cell recapture minimal impact on the cell.

The present invention provides systems for isolating, releasing, and/or capturing cells using LBL films that, in many embodiments, are biodegradable films. Among other things, provided technologies permit investigations of cell properties present in and/or characteristic of specific cell populations, and furthermore allows determinations of such properties with an accuracy that has been difficult to achieve by other means.

In an example embodiment, the present invention is a device comprising a substrate having a surface, and a layer-by-layer (LbL) film disposed on said surface, the LbL film comprising at least one multilayer unit including at least a first layer and a second layer non-covalently associated with one another, wherein at least one of the first layer or the second layer of the at least one multilayer unit is decomposable, and at least one of the first layer or the second layer of the at least one multilayer unit comprises a binding agent that binds to an entity so as to retain the entity in association with the device.

In another embodiment, the present invention is a method of capturing a target entity, comprising the steps of: providing a device comprising a substrate having a surface, and a layer-by-layer (LbL) film disposed on said surface, the LbL film comprising at least a first layer and a second layer non-covalently associated with one another, wherein at least one of the first layer or the second layer of the at least one multilayer unit is decomposable, and at least one of the first layer or the second layer of the at least one multilayer unit comprises a binding agent that binds to the target entity so as to retain the target entity in association with the device, and exposing the device to a sample comprising the target entity so that the target entity is retained in or on the device.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1(a) illustrates a schematic of the architecture of the LbL film and the capture of cells. FIG. 1(b) shows optical florescent images for the modification of films with bioactive molecules and the degradation of films.

FIG. 2 illustrates AFM images of films before and after LbL film degradation.

FIG. 3 demonstrates capture and release of PC3 cells from microstructured chips. The circles shown in the left ‘Capture’ panel indicate the captured cells. The circles in the right ‘Release’ panel indicate the released cells.

FIG. 4 (a) shows a schematics of capturing and releasing of CTCs using microfluidic HB chips modified with enzymatically degradable LbL nano coatings. FIG. 4 (b) the top image shows the structure of HB chip and the bottom image shows fluorescent microscope imaging the surface of HB chip modified with of LbL film and the antibody linkages. The scale bar is 100 μm.

FIGS. 5 (a)-(c) demonstrate[[s]] optimization of film compositions. FIG. 5 (a) shows the name of molecular structures of anionic polymers and cationic polymers used. FIG. 5 (b) illustrates optical fluorescent images of LbL films before and after degradation. FIG. 5 (c) demonstrates fluorescent intensity (FI) change of the films before and after degradation. Fl of all films were normalized by the intensity of ALG/LMWC before degradation. Bars outlined in black represent FI of films before degradation, bars outlined in gray represent FI of files after degradation. White and gray bars represent FI of series of films made by ALG or HA, respectively.

FIGS. 6 (a)-(e) illustrate[[s]] capture and release of spiked PC3 cells from serum free media. FIG. 6 (a) shows optical microscope images for PC3 cells that were captured, during release, and after release. Captured cells are indicated by blue circles, and released cells by red circles. The scale bar is 200 μm. FIG. 6 (b) illustrates release efficiency at various degradation time and flow rates of enzyme solution. FIG. 6 (c) shows film thickness for various film compositions. FIG. 6 (d) illustrates live/dead cell viability test of captured PC3 cells in the HB chip. Scale bar is 200 μm. FIG. 6 (e) illustrates fluorescent microscope images of released PC3 cells that show small pieces of LbL films on the cell surface. The LbL film was made by FITC-labeled PAH and CY5-labeled avidin linker. The cells are stained with DAPI. The scale bar is 10 μm.

FIGS. 7 (a)-(c) demonstrates optical microscope images for PC3 cells that were captured, during release, and after release. Captured cells are indicated by blue circles, and released cells by red circles. The scale bar is 200 μm. FIG. 7 (d) shows release efficiency at various degradation time and flow rates of enzyme solution. FIG. 7 (e) demonstrates live/dead cell viability test of captured PC3 cells in the HB chip. Scale bar is 200 μm. FIG. 7 (f) shows fluorescent microscope images of released PC3 cells that show small pieces of LbL films on the cell surface. The LbL film was made by FITC-labeled PAH and CY5-labeled avidin linker. The cells are stained with DAPI. The scale bar is 10 μm.

FIG. 8 (a) shows released cells were evaluated for viability using a fluorescent LIVE (green)/DEAD (red) assay. PC3 cells were pre-stained with cell tracker orange before spiking into blood. Live PC3 cells shown co-localization of orange color and green color. The scale bars are 100 μm in the main image and 10 μm for the zoom-in image, respectively. FIG. 8 (b) demonstrates cell viability of PC3 cells spiked in blood. Black bars shown the control cells and blue bars shown captured and released cells. FIG. 8 (c) illustrates immunofluorescent staining of cell surface receptors for captured PC3 cells in the HB chip. FIG. 8 (d) shows released PC3 cells. FIG. 8 (e) shows white blood cells with EpCAM expression in green, DAPI nuclear staining in blue, and CD45 expression in red. The scale bars are 20 μm. FIG. 8 (f) illustrates released PC3 cells showing proliferation and maintaining viability for culturing, image was taken after five days of releasing from HB chip. The scale bar is 100 μm.

FIGS. 9 (a)-(d) illustrate[[s]] immunofluorescent staining of cell surface receptors from the HB chip. FIG. 9 (a) shows captured CTC cells. FIG. 9 (b) illustrates released CTC cells from patient blood samples in the HB chip. FIGS. 9 (c) and (d) shown EpCAM, HER2 and MET expression in green, DAPI nuclear staining in blue, and CD45 expression in red. Scales bars are 10 μm.

FIG. 10 shows capture and release of heterogeneous CTCs.

FIG. 11 illustrates PC3 cell capture and release in single microfluidic device.

FIGS. 12a and 12b demonstrate PC3 cells capture in HB chips.

### DETAILED DESCRIPTION OF THE INVENTION

A description of example embodiments of the invention follows.

### Glossary

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an l-amino acid. “Standard amino acid” refers to any of the twenty standard l-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In certain embodiments, the animal is susceptible to infection by influenza. In some embodiments, an animal may be a transgenic animal, genetically engineered animal, and/or a clone.

Antibody agent: As used herein, the term “antibody agent” refers to an agent that specifically binds to a particular antigen. In some embodiments, the term encompasses any polypeptide with immunoglobulin structural elements sufficient to confer specific binding. Suitable antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody: As is known in the art, an “antibody” is an immunoglobulin that binds specifically to a particular antigen. The term encompasses immunoglobulins that are naturally produced in that they are generated by an organism reacting to the antigen, and also those that are synthetically produced or engineered. An antibody may be monoclonal or polyclonal. An antibody may be a member of any immunoglobulin class, including any of the human classes: IgG, IgM, IgA, and IgD. A typical immunoglobulin (antibody) structural unit as understood in the art, is known to comprise a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. Each variable region is further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CH1, CH2 and CH3). In some embodiments, the term “full length” is used in reference to an antibody to mean that it contains two heavy chains and two light chains, optionally associated by disulfide bonds as occurs with naturally-produced antibodies. In some embodiments, an antibody is produced by a cell. In some embodiments, an antibody is produced by chemical synthesis. In some embodiments, an antibody is derived from a mammal. In some embodiments, an antibody is derived from an animal such as, but not limited to, mouse, rat, horse, pig, or goat. In some embodiments, an antibody is produced using a recombinant cell culture system. In some embodiments, an antibody may be a purified antibody (for example, by immune-affinity chromatography). In some embodiments, an antibody may be a human antibody. In some embodiments, an antibody may be a humanized antibody (antibody from non-human species whose protein sequences have been modified to increase their similarity to antibody variants produced naturally in humans). In some embodiments, an antibody may be a chimeric antibody (antibody made by combining genetic material from a non-human source, e.g., mouse, rat, horse, or pig, with genetic material from humans).

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids.

Antigen: An “antigen” is a molecule or entity to which an antibody binds. In some embodiments, an antigen is or comprises a polypeptide or portion thereof. In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or comprises a recombinant antigen.

Aptamer: As used herein, the term “aptamer” means a macromolecule composed of nucleic acid (e.g., RNA, DNA) that binds tightly to a specific molecular target (e.g., an umbrella topology glycan). A particular aptamer may be described by a linear nucleotide sequence and is typically about 15-60 nucleotides in length. Without wishing to be bound by any theory, it is contemplated that the chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For example, aptamers can be modified to dramatically reduce their sensitivity to degradation by enzymes in the blood for use in in vivo applications. In addition, aptamers can be modified to alter their biodistribution or plasma residence time.

Associated: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

Binding agent: In general, the term “binding agent” is used herein to refer to any entity that binds to a target of interest as described herein. In many embodiments, a binding agent of interest is one that binds specifically with its target in that it discriminates its target from other potential binding partners in a particular interaction contact. In general, a binding agent may be or comprise an entity of any chemical class (e.g., polymer, non-polymer, small molecule, polypeptide, carbohydrate, lipid, nucleic acid, etc.). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding moiety partner. In some embodiments, binding agents are or comprise polypeptides (including, e.g., antibodies or antibody fragments). In some embodiments, binding agents are or comprise small molecules. In some embodiments, binding agents are or comprise nucleic acids. In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimotopes. In some embodiments, binding agents are or comprise stapled peptides. In certain embodiments, binding agents are or comprise nucleic acids, such as DNA or RNA.

Biologically active: As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

“Body fluid” refers to any body fluid including, with limitation, serum, plasma, lymph fluid, synovial fluid, follicular fluid, seminal fluid, amniotic fluid, mild, whole blood, sweat, urine, cerebrospinal fluid, saliva, semen, sputum, tears, perspiration, mucus, tissue culture medium, tissue extracts, and cellular extracts. It may also apply to fractions and dilutions of body fluids. The source of a body fluid can be a human body, an animal body, an experimental animal, a plant, or other organism.

Characteristic portion: As used herein, the term “characteristic portion” is used, in the broadest sense, to refer to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Biodegradable: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Detection moiety: As used herein, a “detection moiety” in the context of provided multifunctional agents refers to a molecular structure or module that allows visualization/imaging, measurements (localization, quantification, etc.) and/or monitoring of an agent in vitro and/or in vivo using one or more detection techniques including but not limited to spectroscopic, photochemical, biochemical, immunochemical, electrical, optical, chemical or other means.

Nucleic acid: The term “nucleic acid” as used herein, refers to a polymer of nucleotides. In some embodiments, nucleic acids are or contain deoxyribonucleic acids (DNA); in some embodiments, nucleic acids are or contain ribonucleic acids (RNA). In some embodiments, nucleic acids include naturally-occurring nucleotides (e.g., adenosine, thymidine, guanosine, cytidine, uridine, deoxyadenosine, deoxythymidine, deoxyguanosine, and deoxycytidine). Alternatively or additionally, in some embodiments, nucleic acids include non-naturally-occurring nucleotides including, but not limited to, nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups. In some embodiments, nucleic acids include phosphodiester backbone linkages; alternatively or additionally, in some embodiments, nucleic acids include one or more non-phosphodiester backbone linkages such as, for example, phosphorothioates and 5′-N-phosphoramidite linkages. In some embodiments, a nucleic acid is an oligonucleotide in that it is relatively short (e.g., less that about 5000, 4000, 3000, 2000, 1000, 900, 800, 700, 600, 500, 450, 400, 350, 300, 250, 200, 150, 100, 90, 80, 70, 60, 50, 45, 40, 35, 30, 25, 20, 15, 10 or fewer nucleotides in length).

Polyelectrolyte: The term “polyelectrolyte”, as used herein, refers to a polymer which under some set of conditions (e.g., physiological conditions) has a net positive or negative charge. Polyelectrolytes includes polycations and polyanions. Polycations have a net positive charge and polyanions have a net negative charge. The net charge of a given polyelectrolyte may depend on the surrounding chemical conditions, e.g., on the pH.

Polysaccharide: The term “polysaccharide” refers to a polymer of sugars. Typically, a polysaccharide comprises at least three sugars. In some embodiments, a polypeptide comprises natural sugars (e.g., glucose, fructose, galactose, mannose, arabinose, ribose, and xylose); alternatively or additionally, in some embodiments, a polypeptide comprises one or more non-natural amino acids (e.g, modified sugars such as 2′-fluororibose, 2′-deoxyribose, and hexose).

Reference: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest

Sample: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Small molecule: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

Specific: The term “specific” when used herein with reference to an agent or entity having an activity, is understood by those skilled in the art to mean that the agent or entity discriminates between potential targets or states. For example, an agent is said to bind “specifically” to its target if it binds preferentially with that target in the presence of competing alternative targets. In some embodiments, the agent or entity does not detectably bind to the competing alternative target under conditions of binding to its target. In some embodiments, the agent or entity binds with higher on-rate, lower off-rate, increased affinity, decreased dissociation, and/or increased stability to its target as compared with the competing alternative target(s).

Specificity: As is known in the art, “specificity” is a measure of the ability of a particular ligand to distinguish its binding partner from other potential binding partners.

Substantially: As used herein, the term “substantially” and grammatical equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

Compositions and methods for providing devices comprised of a substrate and a Layer-By-Layer (LBL) film coated on at least a surface of the substrate, which the LBL film comprises binding agents that specifically interact with cells. Such devices are useful, for example, in various cell isolation applications. Among other advantages, such devices permit isolation and release (e.g., via layer degradation) of cells under mild conditions. Those skilled in the art will appreciate that certain aspects of the present disclosure may find application in a variety of contexts, including in isolation of entities other than cells (e.g., of viruses, of cellular components, etc.), and will understand that such embodiments may be encompassed within the present disclosure and claims even if not specifically called out or exemplified.

LBL Films

As is known in the art, “LBL films” are films assembled by serial application of individual layers that associate with one another through non-covalent interactions. LBL technology has proven to be robust and broadly applicable to a variety of different materials (particularly polymers, and most particularly polyelectrolytes).

Those skilled in the art are aware that LBL films may be constructed to have any of a variety of film architectures, (e.g., number of layers, thickness of individual layers [understanding that “merging” of layer materials may occur once films are assembled], overall film thickness, etc.). Moreover, those skilled in the art will appreciate that, as noted above, LBL technology has proven to be applicable to a variety of materials, so that chemical identity of film layers may readily be selected or adjusted by the practitioner. Still further, those skilled in the art will be aware of technologies for incorporating any of a variety of agents into or onto films.

In general, LBL films comprise multiple layers. In many embodiments, LBL films are comprised of multilayer units; each unit comprising individual layers. In accordance with the present disclosure, individual layers in an LBL film interact with one another. In particular, a layer in an LBL film comprises an interacting moiety, which interacts with that from an adjacent layer, so that a first layer associates with a second layer adjacent to the first layer, each contains at least one interacting moiety.

In some embodiments, adjacent layers are associated with one another via non-covalent interactions. Exemplary non-covalent interactions include, but are not limited to, hydrogen bonding, affinity interactions, metal coordination, physical adsorption, host-guest interactions, hydrophobic interactions, pi stacking interactions, hydrogen bonding interactions, van der Waals interactions, magnetic interactions, dipole-dipole interactions and combinations thereof.

LBL films may be comprised of multilayer units with alternating layers of opposite charge, such as alternating anionic and cationic layers. In some embodiments, an interacting moiety is a charge, positive or negative. In some embodiments, an interacting moiety is a hydrogen bond donor or acceptor. In some embodiments, an interacting moiety is a complementary moiety for specific binding such as avidin/biotin. In various embodiments, more than one interaction can be involve in the association of two adjacent layers. For example, an electrostatic interaction can be a primary interaction; a hydrogen bonding interaction can be a secondary interaction between the two layers.

In some embodiments, the present invention provides the insight that at least some potential layer materials, including potential agents for delivery that could otherwise be utilized as layer materials do not and/or cannot carry sufficient charge to mediate stable electrostatic interactions. In addition to electrostatic interaction or alternatively, they can be associated via non-electrostatic interaction in a coated device in accordance with the present invention.

According to the present disclosure, LBL films may be comprised of one or more multilayer units. In some embodiments, an LBL film include a plurality of a single unit (e.g., a bilayer unit, a tetralayer unit, etc.). In some embodiments, an LBL film is a composite that include more than one units. For example, more than one unit can have different film materials (e.g., polymers), film architecture (e.g., bilayers, tetralayer, etc.), film thickness, and/or agents that are associated with one of the units. In some embodiments, an LBL film is a composite that include more than one bilayer units, more than one tetralayer units, or any combination thereof. In some embodiments, an LBL film is a composite that include a plurality of a single bilayer unit and a plurality of a single tetralayer unit. In some embodiments, the number of multilayer units is 3, 5, 10, 20, 30, 40, 50, 60, 70, 80, 90, 100, 150, 200, 300, 400 or even 500.

LBL films may have various thickness depending on methods of fabricating and applications. In some embodiments, an LBL film has an average thickness in a range of about 1 nm and about 100 μm. In some embodiments, an LBL film has an average thickness in a range of about 1 μm and about 50 μm. In some embodiments, an LBL film has an average thickness in a range of about 2 μm and about 5 μm. In some embodiments, the average thickness of an LBL film is or more than about 1 nm, about 5 nm, about 10 nm, about 20 nm, about 50 nm, about 75 nm, about 100 nm, about 200 nm, about 300 nm, about 400 nm, about 500 nm, about 600 nm, about 700 nm, about 800 nm, about 900 nm, about 1 μm, about 1.5 μm, about 2 μm, about 3 μm, about 4 μm, about 5 μm, about 10 μm, about 20 μm, about 50 μm, about 100 μm. In some embodiments, an LBL film has an average thickness in a range of any two values above.

An individual layer of an LBL film can contain a polymeric material. In some embodiment, a polymer is a polypeptide. In some embodiments, a polymer has a relatively small molecule weight. Polymers used herein and in accordance with the present disclosure generally can be biologically derived or natural. Polymers may include charged polysaccharides. In some embodiments, polysaccharides include glycosaminoglycans such as heparin, chondroitin, dermatan, hyaluronic acid, etc. (Some of these terms for glycoasminoglycans are often used interchangeably with the name of a sulfate form, e.g., heparan sulfate, chondroitin sulfate, etc. In some embodiments, polysaccharides include alginic acid, a polysaccharide is composed of two types of uronic acid units, mannuronic acid and guluronic acid. It is intended that such sulfate forms are included among a list of exemplary polymers used in accordance with the present invention). Additionally or alternatively, polymers can be a natural acid. In some embodiments, a polymer is synthetic.

LBL films can be decomposable. In some embodiments, a polymer is degradable or non-degradable. In some embodiments, a polymer is a polyelectrolyte. In many embodiments, a polymer of an individual layer includes a degradable polyelectrolyte. In some embodiments, decomposition of LBL films is characterized by substantially sequential degradation of at least a portion of the polyelectrolyte layers that make up LBL films. Degradation may be at least partially hydrolytic, at least partially enzymatic, at least partially thermal, and/or at least partially photolytic. Degradable polyelectrolytes and their degradation byproducts may be biocompatible so as to make LBL films amenable to use in vivo.

LbL films may be exposed to a liquid medium (e.g., intracellular fluid, interstitial fluid, blood, intravitreal fluid, intraocular fluid, gastric fluids, etc.). In some embodiments, an LbL film comprises at least one polycationic layer that degrades and at least one polyanionic layer that delaminates sequentially. Releasable agents are thus gradually and controllably released from the LbL film. It will be appreciated that the roles of the layers of an LbL film can be reversed. In some embodiments, an LbL films comprises at least one polyanionic layer that degrades and at least one polycationic layer that delaminates sequentially. Alternatively, polycationic and polyanionic layers may both include degradable polyelectrolytes.

Degradable polyelectrolytes can be used in an LBL film disclosed herein, including, but not limited to, hydrolytically degradable, biodegradable, thermally degradable, enzymatically degradable (e.g. hydrolysis or oxidative degradation catalyzed by enzymes), and photolytically degradable polyelectrolytes. Hydrolytically degradable polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Those skilled in the art will recognize that this is an exemplary, not comprehensive, list of biodegradable polymers. Of course, co-polymers, mixtures, and adducts of these polymers may also be employed.

In accordance with some embodiments of the present disclosure, LbL films can be used as a release layer. In many embodiments, a release layer comprised of one or more LbL films are assembled and/or deposited on a substrate. When a release layer is removed from a substrate, for example, by dissolution in a liquid medium, photodegradation, or other equivalent method known in the art, LbL films outside the release layer will be released from the substrate. In addition, any other films or layers coating the substrate may be released.

In various embodiment, a release layer is or comprises a polymer. Such a polymer, in some embodiments, is stable during deposition/assembly and can be converted to become unstable when exposed in a liquid medium for releasing. A release layer in accordance with the present invention comprises one or more multilayer films coated on at least one surface of a substrate. In various embodiments, the LbL release layer comprises a polymer exemplified above. In some embodiments, the polymer is a polysaccharide. In some embodiments the polysaccharide is alginate.

In some embodiments, a polymer can be a photocleavable polymer. Exposing a photocleavable polymer LbL release layer to UV will photocleave hydrophobic moieties. A photocleaved polymer LbL can be pH sensitive, so that the photocleaved polymer LbL is stable at a predetermined pH or in a predetermined pH range, but unstable in different pH range.

Anionic polyelectrolytes may be degradable polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself. Cationic polyelectrolytes may be degradable polymers with cationic groups distributed along the polymer backbone. Cationic groups, which may include protonated amine, quaternary ammonium and phosphonium-derived functions, poly(L-lactide-co-L-lysine), poly(serine ester), poly(4-hydroxy-L-proline ester), poly[α-(4-aminobutyl)-L-glycolic acid], and/or poly([β-amino ester)s or other positively charged or ionizable groups, may be disposed in side groups pendant from the backbone, may be attached to the backbone directly, or can be incorporated in the backbone itself.

In some embodiments, an LBL film comprises at least one polycationic layer that degrades and at least one polyanionic layer that delaminates sequentially. It will be appreciated that the roles of the layers of an LBL film can be reversed. In some embodiments, an LBL films comprises at least one polyanionic layer that degrades and at least one polycationic layer that delaminates sequentially. Alternatively, polycationic and polyanionic layers may both include degradable polyelectrolytes.

In other embodiments, polyanionic and/or polycationic layers may include a mixture of degradable and non-degradable polyelectrolytes. Any non-degradable polyelectrolyte can be used. Exemplary non-degradable polyelectrolytes that could be used in thin films include poly(styrene sulfonate) (SPS), poly(acrylic acid) (PAA), linear poly(ethylene imine) (LPEI), poly(diallyldimethyl ammonium chloride) (PDAC), and poly(allylamine hydrochloride) (PAH).

Alternatively or additionally, the degradation rate may be fine-tuned by associating or mixing non-biodegradable, yet biocompatible polymers with one or more of the polyanionic and/or polycationic layers. Suitable non-biodegradable, yet biocompatible polymers are well known in the art and include polystyrenes, certain polyesters, non-biodegradable polyurethanes, polyureas, poly(ethylene vinyl acetate), polypropylene, polymethacrylate, polyethylene, polycarbonates, and poly(ethylene oxide).

Moreover, the composition of the polyanionic and polycationic layers can be fine-tuned to adjust the degradation rate of each layer within the film. For example, the degradation rate of hydrolytically degradable polyelectrolyte layers can be decreased by associating hydrophobic polymers such as hydrocarbons and lipids with one or more of the layers. Alternatively, the polyelectrolyte layers may be rendered more hydrophilic to increase their hydrolytic degradation rate.

Capture and Release

A variety of methods exist for the capture and subsequent release of various cell types. Methods may be separated into macroprocesses and microprocesses. Macroprocesses includes density gradient centrifugation, microfiltration, and use of antibody-modified magnetic beads. Wherein, for example, captured cells are released from the magnetic beads using trypsin after the beads have been collected by a magnetic sweeper. Microprocesses includes use of lectin-modified microposts, DNA aptamers attached to silicon nanowires, antibody-modified DNA biochips, antibody-modified photolabile linkers on glass substrates, APBA-functionalized multiwalled carbon nanotubes films, interaction between calmodulin with a calmodulin-binding peptide in the presence of calcium, and cryogels. Whereby captured cells are released from introduction of the specific lectin's inhibiting sugar, introduction of exonuclease, enzyme cleavage at the incorporated restriction site, ultraviolet exposure, introduction of fructose, calcium chelation, and mechanical deformation of the cryogel, respectively.

One target of cell capture and release are circulating tumor cells (CTCs), which are cancer cells in the blood stream from primary or metastatic tumor sites in patients with epithelial cancers. They are difficult to isolate and preserve, as there is only one CTC per every billion red blood cells. However, capturing and releasing viable CTCs is an important step for studying cells and cell response in vitro as a potential alternative to invasive biopsies.

Current methods for capturing CTCs include using the differing size and deformability of the CTCs versus blood cells (microfluidic devices with obstacles that sort cells, inertial forces within a flow channel), and using antibody or aptamer modifications to various substrate surfaces, such as in microchannels, on microposts, on gold microelectrodes, on hydrogels, or on thermoresponsive surfaces. The current release methods for the former subset include manual pipetting or separation into different wells, while the those for the latter subset include treatment with trypsin, shear force via media flow, application of a reductive potential, dissolution with a chelator, or alteration of temperature, respectively.

The present invention encompasses the recognition that the existing capture and release method suffer from several issues. Capture and release processes must be improved upon if the full potential of CTCs as a diagnostic and analytic tool is to be realized. Capture and release process improvements addressed by the present invention include the following. First, techniques such as microfiltration often cause damage to the cells. To be fully beneficial the resulting cells must be viable, that is, the cell separation method must not physically harm the cells. Second, certain stresses, such as the enzymatic digestion of trypsin, shear force, temperature variance, and UV exposure, are known to alter the phenotype of captured cells. To attain complete diagnostic and analytic capability, the phenotype of the cells must be preserved. Third, the method must achieve both high cell recovery as well as high cell purity. Fourth, the method must be feasible for disposable point-of-care use, that is, it must not require excessive lab equipment, limiting electrical and optical means of cell detachment.

The structures and methods of the present invention, construction and degradation of LBL polyelectrolyte films within microfluidic devices is a robust method selectively isolating cells from bulk fluid and which can be degraded on command, isolation, and recapture of cells with minimal impact on the cell is possible. These structures and methods provide the capability to investigate the properties of specific cell populations with an accuracy that has been difficult to achieve by other capture and release means. LBL films according to the present invention are particularly useful as they are available from a broad range of materials.

In accordance with some embodiments of the present disclosure, LBL films can be used as a release layer. In many embodiments, a release layer comprised of one or more LBL films are assembled and/or deposited on a substrate. When a release layer is removed from a substrate, for example, by dissolution in a liquid medium, photodegradation, or other equivalent method known in the art, LBL films outside the release layer will be released from the substrate. In addition, any other films or layers coating the substrate may be released.

In various embodiments, a release layer is or comprises a degradable polymer LBL as described above. Such a polymer, in some embodiments, is stable during deposition/assembly and can be converted to become unstable when exposed in a liquid medium for releasing. A release layer in accordance with the present invention comprises one or more multilayer films coated on at least one surface of a substrate. In various embodiments, the LBL release layer is or comprises a polymer exemplified above. In some embodiments, the polymer is or comprises a polysaccharide. In some embodiments the polysaccharide is or comprises alginic acid hyaluronic acid. In some embodiments, the polysaccharide is alginate.

In some embodiments, a polymer can be a photocleavable polymer. Exposing a photocleavable polymer LBL release layer to UV will photocleave hydrophobic moieties. A photocleaved polymer LBL can be pH sensitive, so that the photocleaved polymer LBL is stable at a predetermined pH or in a predetermined pH range, but unstable in different pH range.

According to various embodiments, exposing a release layer to an enzyme lyase will cause the release layer to degrade. In some embodiments, the LBL layer may be pH sensitive, so that the enzyme lyased polymer LBL film is stable at a predetermined pH or in a predetermined pH range, but unstable in different pH range. In various embodiments an predetermined pH range is optimal between the range of about pH 1-2, pH 2-3, pH 3-4, pH 4-5, pH 5-6, or even 6-7 and higher. As a result of exposure of the release layer to an enzyme lyase, any portion of the LBL remaining is separated from the substrate.

Binding Agents

In some embodiments of the present invention, one or more layers of an LBL film includes a binding agent. In particular, the binding agents of the present invention target (i.e., binds specifically to) one or more cellular components or markers (e.g. surface proteins, glycoprotein, group of proteins, carbohydrates, antigens, immunoglobulin, receptors, among others for distinguishing a cell or subset of cells from another defined subset of cells) so that, when a device as described herein (e.g., comprising a substrate having an LBL film coated on part or all of its surface) is contacted with a sample containing appropriate cells (e.g., cells containing or expressing, particularly on their surface, a component or marker targeted by the binding agent), the cells are retained in or on the film.

As described herein, in some embodiments, a binding agent may be a single chemical entity. In some embodiments, a binding agent may be a complex of two or more chemical entities noncovalently (and sometimes) reversibly associated with one another. For example, in some embodiments, a binding agent may be “modular” in that it comprises a generic interacting component and a specific targeting component.

Typically, an LBL film layer will contain a plurality of binding agents. In some embodiments, an LBL film layer contains a plurality of the same binding agent. In some embodiments, different layers contain the same binding agent. In some embodiments, more than one layer of an LBL film contains a plurality of binding agents. In some embodiments, an LBL layer contains a plurality of different binding agents (e.g., targeting different cell components or markers). In various embodiments, a binding agent is attached to (or otherwise associated with) the outermost layer of an LBL film. Alternatively or additionally, a binding agent may be attached to or otherwise associated with one or more other layers of the film.

In some embodiments, a binding agent provides attachment between cells and an LBL film release layer. Typically, a binding agent associates with its target through non-covalent interactions. According to some embodiments, LBL may bond to a target through a conjugated protein bond, for example, a biotinylated polysaccharide. In some embodiments of the present invention, for example, biotin, avidin, streptavidin, and/or neutravidin may be used to provide binding activity. To give but one particular example that will illustrate for those skilled in the art, in some embodiments, a modular binding agent comprises a generic interacting pair (e.g., such as biotin/avidin or streptavidin) or a class-specific antibody and an antibody constant region of the relevant class), where one member of the pair is associated with an LBL film layer and the other is associated with the specific targeting component (e.g., antibody, aptamer, or lectin that interacts with appropriate specificity and affinity with a particular target of interest).

In some embodiments, binding agents are characterized in that they interact specifically with cells under conditions of contact, so that cells become bound to and/or otherwise retained in or on the LBL film. The binding agent provides specificity between cells and the release layer. To provide attachment for the cells, in various embodiments, a conjugated protein bond provides a link between the release layer and a structure having specificity and/or affinity for a particular cell or cell type, for example, an antibody. According to some embodiments of the present invention, the binding agent utilizes antibody-antigen interactions in combination with multilayer LBL films to capture cells. Without wishing to be bound by any particular theory, it is proposed that the binding agent(s) present in a particular film provide(s) the desired attachment and selectivity for isolation of cells by the device.

A cell is an exemplary embodiment of an agent for capture. Other non-limiting examples of agents that may be desired for capture include, cell structures, small molecules, viruses, and nucleic acids. LBL films may be exposed to a liquid medium (e.g., intracellular fluid, interstitial fluid, blood, intravitreal fluid, intraocular fluid, gastric fluids, etc.).

In some embodiments, cells are retained on or in the outermost layer of an LBL film. In some embodiments, cells are retained in or on a degradable layer of the LBL film. Alternatively or additionally, in some embodiments, cells are retained in or on one or more other layers, including preferably one or more other degradable layers, of an LBL film. In many embodiments, specificity between an antigen of the cells and the antibody results in capture of a specific type of cell, a desired cell.

Substrates

According to various embodiments of the present invention, an LBL film as described herein is applied to or assembled on a substrate so that it is bound to or adhered to the substrate. Those of ordinary skill in the art will appreciate that a substrate for use in accordance with the present invention may be or comprise any of a variety of materials. In some embodiments, a substrate may comprise more than one material to form a composite. In some embodiments, such material is or comprises a metal (e.g., gold, silver, platinum, and aluminum); metal-coated materials; metal oxides, or combinations thereof; alternatively or additionally, in some embodiments, such material is or comprises plastics, ceramics, silicon, glasses, mica, graphite, urethanes, rubber, molded plastic, polymethyl-methacrylate (PMMA), polycarbonate, polytetrafluoroethylene (TEFLON), polyvinylchloride (PVC), polymethylsiloxane (PDMS), and polysulfone or combination thereof.

In some embodiments, the substrate material is a polymer or co-polymer. Persons of ordinary skill in the art would be aware that exemplary polymers include, but are not limited to, polyamides, polyphosphazenes, polypropylfumarates, polyethers, polyacetals, polycyanoacrylates, polyurethanes, polycarbonates, polyanhydrides, polyorthoesters, polyhydroxyacids, polyacrylates, ethylene vinyl acetate polymers and other cellulose acetates, polystyrenes, poly(vinyl chloride), poly(vinyl fluoride), poly(vinyl imidazole), poly(vinyl alcohol), poly(ethylene terephthalate), polyesters, polyureas, polypropylene, polymethacrylate, polyethylene, poly(ethylene oxide)s and chlorosulphonated polyolefins; and combinations thereof.

In some embodiments, the substrate is made glass with a functionalize microfluidic chip bonded thereon. Those of ordinary skill would understand, that the microfluidic chip for use in accordance with the present invention may be or comprise any of a variety of materials. In various embodiments the microfluidic chip material is or comprises, but is not limited to polydimethysiloxane (PDMS), glass, polymer, co-polymer, urethanes, rubber, molded plastic, polymethyl-methacrylate (PMMA), polycarbonate, polytetrafluoroethylene (TEFLON), polyvinylchloride (PVC), polymethylsiloxane (PDMS), and polysulfone.

### Other Components

In some embodiments, devices as provided by the present invention include one or more additional components other than the substrate and LBL film (containing the capture agent). For example, in some embodiments, a device will include one or more payload entities such as a a therapeutic entity or a detectable entity. In some embodiments, such a payload entity is associated with one or more layers of the LBL film, and may be released from the device via layer degradation (or delamination).

There is a particular interest in achieving delivery of payload entities such as vaccines and/or therapeutic agents. Such agents may be associated with the LBL films disclosed herein. In various embodiments, these agents may be released. One of ordinary skill in the art would appreciate therapeutic agents would include, for example, antibiotics, NSAIDs, glaucoma medications, angiogenesis inhibitors, neuroprotective agents). Moreover, other therapeutic agents may be associated with the LBL films, including, for example, cytotoxic agents, diagnostic agents (e.g. contrast agents; radionuclides; and fluorescent, luminescent, and magnetic moieties), prophylactic agents (e.g. vaccines), transfection agents, immunological agents (e.g., adjuvant), nutraceutical agents (e.g. vitamins, minerals, etc.), and/or other substances that may be suitable for introduction to biological tissues, including pharmaceutical excipients and substances for tattooing, cosmetics, and the like.

In some embodiments, therapeutic agents can be small molecules, large (i.e., macro-) molecules, or a combination thereof. Exemplary agents include, but are not limited to, small molecules, nucleic acids (e.g., siRNA, RNAi, and microRNA agents), proteins (e.g. antibodies), peptides, lipids, carbohydrates, hormones, metals, radioactive elements and compounds, drugs, vaccines, transfection agents, immunological agents, and/or organic compound with pharmaceutical activity or a clinically-used drug, etc., and/or combinations thereof. In some embodiments, an agent can be a drug formulation including various forms, such as liquids, liquid solutions, gels, hydrogels, solid particles (e.g., microparticles, nanoparticles), or combinations thereof.

In some embodiments, a therapeutic agent is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, etc.

In some embodiments, a therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g., penicillin and clavulanic acid).

In some embodiments, a therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof.

In some embodiments, a therapeutic agent may be an anti-inflammatory agent. Anti-inflammatory agents may include corticosteroids (e.g., glucocorticoids), cycloplegics, non-steroidal anti-inflammatory drusg (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

Those skilled in the art will recognize that this is an exemplary, not comprehensive, list of therapeutical agents that can be released using compositions and methods in accordance with the present disclosure.

There is a particular interest in achieving delivery of payload entities such as detectable entity. Detectable agents/entities may be associated with the LBL films disclosed herein. Typically, these agents are associated with a structure over which monitoring is desired. Those skilled in the art will appreciate detectable agents that can be used with the structures and methods in accordance with the present disclosure. In some embodiments, a detectable payload entity permits monitoring of device intactness, stability, localization, etc. For example, detection of a detectable entity associated with a particular layer of an LBL film may be utilized to detect, monitor, and/or characterize extent and/or timing of assembly and/or degradation of the relevant layer. In various embodiments, these agents fluoresce, thereby permitting visualization and confirmation that the film was correctly built and/or modified, for example, through avidin or EpCAM modifications. Additionally, according to some embodiments, whether an enzyme lyase, for example, alginate lyase, properly degraded the LBL film after enzyme exposure may be confirmed using such a detectable agent. According to some embodiments, fluorescent images of LbL films may be acquired both before and after degradation. Recording fluorescent intensity and accounting for a change of fluorescent intensity may indicate film composition before and after degradation, using, for example Streptavidin Dylight 650 fluorescent conjugates and/or fluorescein isothiocyanate (FITC). In some embodiments, fluorescent microscope images of detectable agents may show the surface of HB chip modified with of LbL film and the antibody linkages. In various embodiments, fluorescent microscope images of released cells show small pieces of LbL films on the cell surface confirming cell capture and release. Using a Live/Dead fluorescent assay, in some embodiments recovered cell viability can be measured.

Microfluidic Chip

In some embodiments, a microfluidic chip is utilized to assemble, monitor, and/or characterize devices as provided by the present invention.

The width and height of the microchannel may vary from tens of micrometers to hundreds of micrometers, depending on the application, while the length of the microchannel is typically from a few millimeters to tens of millimeters. The channel connects two openings, used as inlet and outlet reservoirs for the delivery of solutions of the present invention.

Such a device can be placed on top of and/or bonded to a negatively charged surface of a flat or non-flat substrate as disclosed above. An initial substrate surface charge can be created by oxygen plasma treatment of the surface, which also sterilizes the surface.

A volume of a channel can vary depending on a sample size (e.g., coating material) and/or a particular application. In some embodiments, the volume of a channel may be about or less than 100 μL, 50 μL, 10 μL, 1 μL, 500 nL, 200 nL, 100 nL, 90 nL, 80 nL, 70 nL, 60 nL, 50 nL, 40 nL, 30 nL, 20 nL, 10 nL, 5 nL, 2 nL, or even 1 nL. In some embodiments, the volume of a channel may be in a range of about 1000 μL to about 1 nL, about 10 μL to about 5 nL, about 100 nL to about 10 nL or any two values above.

Delivery of sample as described here may be through the microchannels. In some embodiments, the rates of flow of the solutions may be about or less than 0.1 mL/hour, 0.2 mL/hour, 0.3 mL/hour, 0.4 mL/hour, 0.5 mL/hour, 0.6 mL/hour, 0.7 mL/hour, 0.8 mL/hour, 0.9 mL/hour, 1.0 mL/hour, 1.1 mL/hour, 1.2 mL/hour, 1.3 mL/hour, 1.4 mL/hour, 1.5 mL/hour, 1.6 mL/hour, 1.7 mL/hour, 1.8 mL/hour, 1.9 mL/hour, 2.0 mL/hour, 2.1 mL/hour, 2.2 mL/hour, 2.3 mL/hour, 2.4 mL/hour, 2.5 mL/hour, 2.6 mL/hour, 2.7 mL/hour, 2.8 mL/hour, 2.9 mL/hour, 3.0 mL/hour, 3.1 mL/hour, 3.2 mL/hour, 3.3 mL/hour, 3.4 mL/hour, 3.5 mL/hour, 3.6 mL/hour, 3.7 mL/hour, 3.8 mL/hour, 3.9 mL/hour, 4.0 mL/hour, 4.1 mL/hour, 4.2 mL/hour, 4.3 mL/hour, 4.4 mL/hour, 4.5 mL/hour, 4.6 mL/hour, 4.7 mL/hour, 4.8 mL/hour, 4.9 mL/hour, or even 5.0 mL/hour. In some embodiments, the rates of flow of solutions in the channel may be in a range of about 10 mL/hour or above.

Although releasing captured CTCs with alginate lyase has already been demonstrated by dissolving biofunctionalized sacrificial alginate hydrogels, the present structure and methods has the following advantages and improvements over existing methods: (1) can be easily used to uniformly modify microstructures in a closed chamber; (2) provides for noninvasive release, higher selectivity for capture and higher recovery efficiency; (3) results in high viability of the released cells; (4) provides more control over the thickness of the film on the nanometer scale; (5) enables the rapid incorporation of multiple components (e.g. antibodies) into the film architectures; (6) provides for fast release of captured cells by overcoming diffusion limitations (the salt approach to build porous films); and (7) offers the ability to tune the degradation time of the film by changing the pair of polyelectrolytes or other construction parameters during the film preparation.

### Uses

In some embodiments, devices as described herein are utilized to isolate cells from samples, and particularly from biological samples. Those of ordinary skill in the art will readily appreciate that there are a variety of contexts in which such cell isolation is useful. According to some embodiments, this capture and release LBL film technology may be found in modifying current and future medical devices to include or improve cell capture capability. Moreover, in some embodiments, utilization of the present invention may include monitoring cell populations within body fluids or tissues, retrieval of specific cell populations from a bulk cellular milieu, recruitment of particular cell populations to a film surface, and/or the localized enrichment of a distinct cellular population or proteins on a coated surface. To give but a few examples, in some embodiments, provided devices are utilized to isolate cells from samples of a patient who may be suffering from or susceptible to a particular disease, disorder, or condition, and/or who may be receiving a particular therapeutic regimen. In some embodiments, detection, quantitation, and/or characterization of one or more cell types in one or more samples from the patient may facilitate improved treatment and/or understanding of progression of the disease, disorder, or condition and/or effectiveness of treatment. Alternatively or additionally, in some embodiments, provided devices may be utilized to identify, detect, characterize and/or assess one or more potential cell binding agents, for example to assess its utility or effectiveness in targeting a particular cell type, for example for purposes of detection, isolation, and/or treatment. A particular example of such use would be in the capture and controlled release of circulating tumor cells and the isolation of circulating progenitor cells.

In an example embodiment, the invention is a structure, comprising:
a substrate;
a first film layer to a surface of the substrate; and
at least one subsequent film layer applied to the first film layer, or to an outermost subsequent film layer, wherein
   i) each subsequent film layer interacts with the film layer to which it is applied via a non-covalent association reaction selected from the group consisting of: affinity interactions, metal coordination, physical adsorption, host-guest interactions, hydrophobic interactions, pi stacking interactions, hydrogen bonding interactions, van der Waals interactions, magnetic interactions, electrostatic interactions, and/or dipole-dipole; and
   ii) at least one film layer comprises a binding agent that specifically binds to cells so as to retain them in association with the device, which binding agent may be incorporated into the at least one film layer prior to or after its incorporation into the device, so that an LBL film is assembled on the surface.

In another embodiment, at least one LBL film is contiguous with the substrate.

In another embodiment, the LBL film is photodegradable.

In another embodiment, the LBL film is biodegradable.

In another embodiment, the surface is the entire surface of the substrate.

In another embodiment, the binding agent captures entities from the group consisting of: cells, cell structures, small molecules, viruses, and nucleic acids.

In another embodiment, the cells are circulating tumor cells (CTCs).

In another embodiment, the CTCs are from a human prostate cancer (PC3) cell line.

In another embodiment, at least one layer is comprised of a polymer.

In another embodiment, each layer is comprised of a polymer.

In another embodiment, at least one layer is comprised of a polyelectrolyte.

In another embodiment, at least two adjacent layers are comprised of polyelectrolytes, the polyelectrolytes being of opposite charge such that association of the adjacent layers with each other can be mediated by charge-charge interactions.

In another embodiment, at least one layer is comprised of an anionic polyelectrolyte selected from the group consisting of carboxylate, sulfonate, sulphate, phosphate, and/or nitrate.

In another embodiment, at least one layer is comprised of a cationic polyelectrolyte selected from the group consisting of: protonated amine, quaternary ammonium and phosphonium-derived functions, poly(L-lactide-co-L-lysine), poly(serine ester), poly(4-hydroxy-L-proline ester), poly[α-(4-aminobutyl)-L-glycolic acid], and/or poly([β-amino ester)s.

In another embodiment, the at least two adjacent polyelectrolyte layers comprise a first anionic layer which is comprised of an anionic polyelectrolyte selected from the group consisting of carboxylate, sulfonate, sulphate, phosphate, and/or nitrate, and a second cationic layer which is comprised of a cationic polyelectrolyte selected from the group consisting of a protonated amine, quaternary ammonium and phosphonium-derived functions, poly(L-lactide-co-L-lysine), poly(serine ester), poly(4-hydroxy-L-proline ester), poly[α-(4-aminobutyl)-L-glycolic acid], and/or poly([β-amino ester)s.

In another embodiment, the first anionic layer is alginate and the second cationic layer is polyallylamine hydrochloride (PAH).

In another embodiment, the binding agent is or comprises a protein.

In another embodiment, the binding agent is or comprises biotin.

In another embodiment, the binding agent is or comprises avidin.

In another embodiment, binding agent is or comprises an antibody.

In another embodiment, the binding agent is or comprises a complex comprised of at least two discrete entities that are physically bound to each other via noncovalent interaction.

In another embodiment, the structure further comprises a detection moiety associated with at least one LBL film.

In another embodiment, the structure further comprises a detection moiety associated with the binding agent.

In another embodiment, the antibody binds with cells using antibody-antigen interactions.

In another embodiment, the antibody is EpCAM.

In another embodiment, the binding agent specifically interacts with a at least one cell surface marker.

In another embodiment, the cell surface marker is selected from surface proteins, glycoprotein, group of proteins, carbohydrates, antigens, immunoglobulin, and/or receptors.

In another embodiment, the cell surface marker is or comprises an antigen specific to EpCAM antibodies of PC3 cells.

In another embodiment, at least one LBL films comprise polyanionic bilayers of alternating alginate and a detection moiety.

In another embodiment, the LBL film comprises a plurality of bilayers.

In another embodiment, the LBL film comprises a plurality of tetralayers.

In another embodiment, each layer is comprised of a polymer.

In another embodiment, every other layer is a same polymer.

In another example embodiment, the present invention is a method of making a biodegradable LBL film, comprising the steps of coating a substrate with at least one LBL film with binding agents associated with the at least one LBL film.

In another example embodiment, the present invention is a method of preparing a device, comprising steps of:
applying a first film layer to a surface of the substrate; and
applying at least one subsequent film layer to the first film layer, or to an outermost subsequent film layer, wherein:
   i) each subsequent film layer interacts with the film layer to which it is applied via a non-covalent association reaction selected from the group consisting of: affinity interactions, metal coordination, physical adsorption, host-guest interactions, hydrophobic interactions, pi stacking interactions, hydrogen bonding interactions, van der Waals interactions, magnetic interactions, electrostatic interactions, and/or dipole-dipole; and
   ii) at least one film layer comprises a binding agent that specifically binds to cells so as to retain them in association with the device, which binding agent may be incorporated into the at least one film layer prior to or after its incorporation into the device, so that an LBL film is assembled on the surface.

In another embodiment, at least one LBL film is contiguous with the surface.

In another embodiment, the surface is the entire surface of the substrate.

In another embodiment, the coating step is performed by serially applying alternating polyelectrolyte layers.

In another embodiment, at least one layer is comprised of a polymer.

In another embodiment, each layer is comprised of a polymer.

In another embodiment, at least one layer is comprised of a polyelectrolyte.

In another embodiment, at least two adjacent layers are comprised of polyelectrolytes, the polyelectrolytes being of opposite charge such that association of the adjacent layers with each other can be mediated by charge-charge interactions.

In another embodiment, the step of applying a subsequent layer is repeated until an LBL film comprised of 3, 5, 10, 20, 30, 40, 50, 60, 70, 80, 90, 100, 150, 200, 300, 400 or even 500 bilayer units is assembled on the surface.

In another embodiment, the at least one LBL film comprises alginate and fluorescent poly(allylamine hydrochloride) (PAH).

In another embodiment, at least one layer is comprised of an anionic polyelectrolyte selected from the group consisting of carboxylate, sulfonate, sulphate, phosphate, and/or nitrate.

In another embodiment, at least one layer is comprised of a cationic polyelectrolyte selected from the group consisting of: protonated amine, quaternary ammonium and phosphonium-derived functions, poly(L-lactide-co-L-lysine), poly(serine ester), poly(4-hydroxy-L-proline ester), poly[α-(4-aminobutyl)-L-glycolic acid], and/or poly([β-amino ester)s.

In another embodiment, the at least two adjacent polyelectrolyte layers comprise a first anionic layer which is comprised of an anionic polyelectrolyte selected from the group consisting of carboxylate, sulfonate, sulphate, phosphate, and/or nitrate, and a second cationic layer which is comprised of a cationic polyelectrolyte selected from the group consisting of a protonated amine, quaternary ammonium and phosphonium-derived functions, poly(L-lactide-co-L-lysine), poly(serine ester), poly(4-hydroxy-L-proline ester), poly[α-(4-aminobutyl)-L-glycolic acid], and/or poly([β-amino ester)s.

In another embodiment, the first anionic layer is alginate and the second cationic layer is polyallylamine hydrochloride (PAH).

In another embodiment, the step of coupling comprises modifying at least one of the alternating polyelectrolyte layers with at least one binding agent.

In another embodiment, the binding agent is or comprises a protein.

In another embodiment, the binding agent is or comprises biotin.

In another embodiment, the binding agent is or comprises avidin.

In another embodiment, the binding agent is or comprises an antibody.

In another embodiment, the binding agent is or comprises a complex comprised of at least two discrete entities that are physically bound to each other via noncovalent interaction.

In another embodiment, the method further comprises a detection moiety for visualizing assembly of the at least one LBL film.

In another embodiment, the antibody binds with cells using antibody-antigen interactions.

In another embodiment, the antibody is EpCAM.

In another example embodiment, the present invention is a method of using a biodegradable LBL film, comprising steps of:
providing a device comprising:
a substrate; and
an LBL film coated on a surface of the substrate, wherein at least one layer of the LBL film comprises at least one binding agent, wherein the at least one binding agent is characterized by an ability to selectively interact with cells so that, when the device is contacted with a sample comprising cells, at least some cells from the sample are retained in or on the device;
exposing the device to a sample comprising cells under conditions and for a time sufficient so that cells are retained in or on the device.

In another embodiment, the step of exposing comprises exposing to a sample of body fluid.

In another embodiment, the body fluid is selected from the group consisting of: serum, plasma, lymph fluid, synovial fluid, follicular fluid, seminal fluid, amniotic fluid, mild, whole blood, sweat, urine, cerebrospinal fluid, saliva, semen, sputum, tears, perspiration, mucus, tissue culture medium, tissue extracts, and cellular extracts.

In another embodiment, the binding agents specifically interact with cells of a particular cell type, so that the retained cells are of that cell type.

In another embodiment, the step of exposing is performed under conditions and for a time sufficient so that cells of the particular cell type are retained and cells of other cell types are not.

In another embodiment, the binding agent specifically interacts with a at least one cell surface marker.

In another embodiment, the cell surface marker is selected from surface proteins, glycoprotein, group of proteins, carbohydrates, antigens, immunoglobulin, and/or receptors.

In another embodiment, the cell surface marker is or comprises an antigen specific to EpCAM antibodies of PC3 cells.

In another embodiment, the LBL films comprise polyanionic bilayers of alternating alginate and a detection moiety.

In another embodiment, the method further comprises a step of separating the device from the sample, thereby isolating the retained cells.

In another embodiment, the method further comprises a step of degrading at least the outermost layer of the LBL film, thereby releasing cells retained in or on that layer.

In another embodiment, the method further comprises a step of degrading at least one additional layer, thereby releasing cells retained in or on that layer.

In another embodiment, at least one step of degrading comprises performing or permitting hydrolytic degradation.

In another embodiment, at least one step of degrading comprises performing or permitting enzymatic degradation.

In another embodiment, the method further comprises a step of analyzing released cells.

In another example embodiment, the present invention is a method comprising steps of:

providing a device comprised of:
a substrate;
an LBL film coated on at least a surface of the substrate, which LBL film comprises:
   a plurality of polymer layers, each associated with its adjacent layers by virtue of non-covalent interactions therebetween;
a test binding agent associated with at least the outermost layer, which the test binding agent is to be assessed for its ability to specifically retain a particular target from a sample;

contacting the device with a sample containing the target; and

assessing extent to which the target is specifically retained in the device.

In another example embodiment, the present invention is a structure, comprising:

a substrate; and

a release layer on the substrate, wherein the release layer comprises at least one layer-by-layer (LbL) film; and

a plurality of binding assemblies, wherein an outer layer of the at least one LbL film is modified with the plurality of binding assemblies.

In another embodiment, the release layer is contiguous with the substrate.

In another embodiment, the release layer is photodegradable.

In another embodiment, the release layer is biodegradable.

In another embodiment, the plurality of binding assemblies are modified to capture cells, cell structures, small molecules, and nucleic acids.

In another embodiment, the cells are circulating tumor cells (CTCs).

In another embodiment, the CTCs are from a human prostate cancer (PC3) cell line.

In another embodiment, the plurality of binding assemblies comprises antibodies bound to the LbL films.

In another embodiment, the antibodies are biotinylated antibodies.

In another embodiment, the antibodies bind with cells using antibody-antigen interactions.

In another embodiment, the release layer is a multilayer LbL polymer films.

In another embodiment, the multilayer LbL polymer films are biotinylated.

In another embodiment, the biotin binding protein is avidin.

In another embodiment, the biotinylated antibodies are biotinylated EpCAM antibodies.

In another embodiment, the biotin binding protein is avidin.

In another embodiment, the biotinylated multilayer LbL polymer films comprise alternating polycationic and polyanionic layers.

In another embodiment, the alternating polycationic and/or polyanionic layers are a degradable polyelectrolyte.

In another embodiment, the degradable polyelectrolyte comprises a polymer selected from polyester, polyanhydride, polyorthoester, polyphosphazene, polyphosphoester, polysaccharide, and any combination thereof

In another embodiment, the polyanionic layers comprises alginate and fluorescent poly(allylamine hydrochloride) (PAH) in alternating bilayers.

In another embodiment, the substrate is glass.

In another embodiment, the structure further comprises a herringbone (HB) microfluidic chip bonded to the glass.

In another embodiment, the HB microfluidic chip comprises a first layer and a second layer, wherein the first layer comprises microfluidic channels and the second layer comprises (HB) structures.

In another example embodiment, the present invention is a method of making a biodegradable layer-by-layer (LbL) film for capture and release, comprising the steps of:

coating a substrate with at least one LbL film; and

coupling a plurality of binding assemblies to the at least one LbL film.

In another embodiment, the at least one LbL film is contiguous with the substrate.

In another embodiment, the coating step is performed by alternating polyelectrolyte layers.

In another embodiment, the at least one LbL film comprises alginate and fluorescent poly(allylamine hydrochloride) (PAH).

In another embodiment, further comprising modifying the alternating polyelectrolyte layers with a binding protein.

In another embodiment, the binding protein is biotin.

In another embodiment, the biotin binding protein is avidin.

In another embodiment, the binding assemblies are antibodies bound to the LbL films by the binding protein.

In another embodiment, the binding protein is biotin.

In another embodiment, the biotin binding protein is avidin.

In another embodiment, the method further comprises a fluorescence marker for visualizing assembly of the at least one LbL film.

In another embodiment, the antibodies bind with cells using antibody-antigen interactions.

In another embodiment, the antibody is EpCAM.

In another example embodiment, the present invention is a method of using a biodegradable LbL film for capture and release; comprising the steps of:

providing a substrate coated with a Layer-by-Layer (LbL) film modified with a plurality of binding assemblies;

exposing the coated substrate to a suspension of cells, wherein at least one cell adheres to at least one of the plurality of binding assemblies; and

separating the coating from the substrate.

In another embodiment, the LbL films comprise polyanionic bilayers of alternating alginate and fluorescent poly(allylamine hydrochloride) (PAH).

In another embodiment, the plurality of binding assemblies comprises antibodies bound to the LbL films.

In another embodiment, the antibodies bind with cells using antibody-antigen interactions.

In another embodiment, the suspension of cells comprise circulating tumor cells (CTCs).

In another embodiment, the CTCs are from a human prostate cancer (PC3) cell line.

In another embodiment, the separating step is performed by degrading the LbL films.

In another embodiment, the degrading step is performed by flowing an enzyme lyase specific to the LbL film.

In another example embodiment, the present invention is a method of capturing a target entity, comprising steps of:
providing a device comprising:
   a substrate having a surface; and
   a layer-by-layer (LbL) film disposed on said surface, the LbL film comprising at least a first layer and a second layer non-covalently associated with one another,
      wherein at least one of the first layer or the second layer of the at least one multilayer unit is decomposable, and at least one of the first layer or the second layer of the at least one multilayer unit comprises a binding agent that binds to the target entity so as to retain the target entity in association with the device; and
exposing the device to a sample comprising the target entity so that the target entity is retained in or on the device.

In another embodiment, the binding agent specifically interacts with at least one cell surface marker.

In another embodiment, the cell surface marker is selected from the group consisting of surface proteins, glycoprotein, group of proteins, carbohydrates, antigens, immunoglobulin, and receptors.

In another embodiment, the cell surface marker associates with EpCAM.

In another embodiment, the first layer and the second layer of the at least one multilayer unit are non-covalently associated with each other by an interaction selected from: affinity interactions, metal coordination, physical adsorption, host-guest interactions, hydrophobic interactions, pi stacking interactions, hydrogen bonding interactions, van der Waals interactions, magnetic interactions, electrostatic interactions, and dipole-dipole interactions.

In another embodiment, the LbL film coats the entire surface of a substrate.

In another embodiment, the binding agent binds to a target entity selected from the group consisting of: cells, small molecules, viruses, and nucleic acids.

In another embodiment, the cells are circulating tumor cells (CTCs).

In another embodiment, the CTCs are human prostate cancer (PC3) cells.

In another embodiment, at least one of the first layer or the second layer of the at least one multilayer unit comprises a polyelectrolyte.

In another embodiment, the first layer comprises a first polyelectrolyte having a first charge, and the second layer comprises a second polyelectrolyte having a second charge, the second charge being opposite to the first change, and the first layer and the second layer being are next to and associated with one another by charge-charge interactions.

In another embodiment, at least one of the first layer or the second layer of the at least one multilayer unit comprises an anionic polyelectrolyte comprising a functional group selected from the group consisting of carboxylate, sulfonate, sulphate, phosphate, and nitrate.

In another embodiment, at least one of the first layer or the second layer of the at least one multilayer unit comprises a cationic polyelectrolyte selected from the group consisting of poly(L-lactide-co-L-lysine), poly(serine ester), poly(4-hydroxy-L-proline ester), poly[α-(4-aminobutyl)-L-glycolic acid], and poly([β-amino ester)s, each of which is optionally substituted with a functional group selected from protonated amine and phosphonium.

In another embodiment, the first layer of the at least one multilayer unit comprises an anionic polyelectrolyte comprising a functional group selected from the group consisting of carboxylate, sulfonate, sulphate, phosphate, and nitrate, and the second layer of the at least one multilayer unit comprises a cationic polyelectrolyte selected from the group consisting of a, poly(L-lactide-co-L-lysine), poly(serine ester), poly(4-hydroxy-L-proline ester), poly[α-(4-aminobutyl)-L-glycolic acid], and poly([β-amino ester)s, each of which is optionally substituted with a functional group selected from protonated amine and phosphonium.

In another embodiment, the first layer of the at least one multilayer unit comprises alginate.

In another embodiment, the second layer of the at least one multilayer unit comprises polyallylamine hydrochloride (PAH).

In another embodiment, the first layer of the at least one multilayer unit comprises alginate and the second layer of the at least one multilayer unit comprises polyallylamine hydrochloride (PAH).

In another embodiment, the binding agent is a protein.

In another embodiment, the binding agent is biotin.

In another embodiment, the binding agent is avidin.

In another embodiment, the binding agent is an antibody or a functional fragment thereof.

In another embodiment, the antibody binds to the cell surface antigen EpCAM.

In another embodiment, the binding agent is a complex comprising at least two discrete entities that are bound to each other via a non-covalent interaction.

In another embodiment, the device further comprises a detection moiety associated with at least one of the first layer or the second layer of the at least one multilayer unit.

In another embodiment, the device further comprises a detection moiety associated with the binding agent.

In another embodiment, the at least one multilayer unit is a bilayer.

In another embodiment, the LbL comprises 3, 5, 10, 20, 30, 40, 50, 60, 70, 80, 90, 100, 150, 200, 300, 400, or 500 bilayer units.

In another embodiment, the at least one multilayer unit is a bilayer, the first layer of the at least one multilayer unit comprises alginate, and the second layer of the at least one multilayer unit comprises a detection moiety.

In another embodiment, the at least one multilayer unit is a tetralayer.

In another embodiment, at least one of the first layer or the second layer is the same in each multilayer unit.

In another embodiment, the device further comprises at least one payload entity.

In another embodiment, the substrate is a microfluidic chip.

### EXAMPLES

### Example 1

### Exemplary Operational Considerations and Methods

The following is an example of the building of a biodegradable LBL film for the capture and release of circulating tumor cells (CTCs) in blood from microstructured chips. In this test human prostate cancer (PC3) cell line were used as model cells. This method can be generalized for the construction of films with various components and for the capture of many different cell types.

Materials

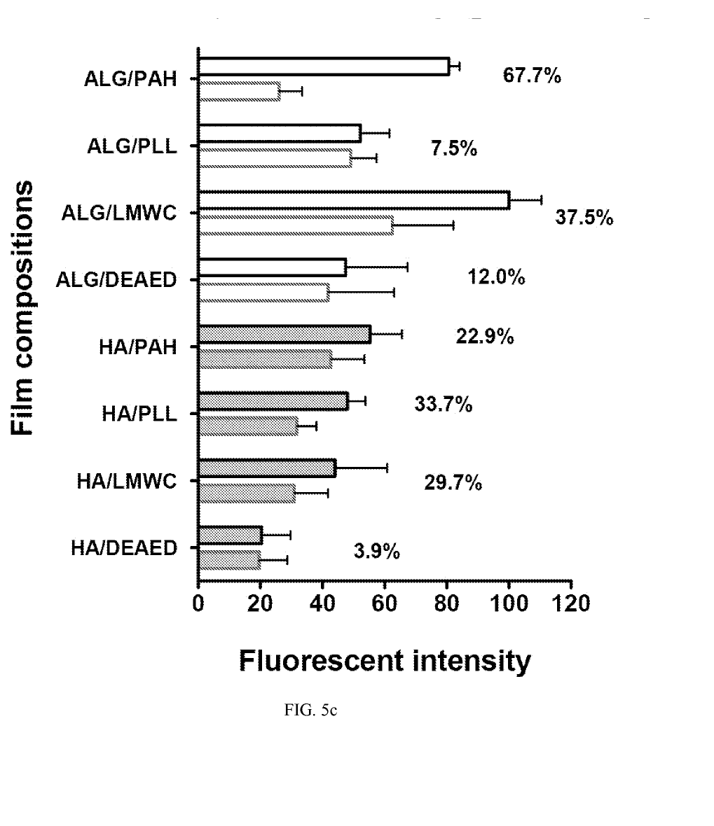

According to various embodiments, substrate, microchip, polymer or polyelectolyte materials used include: Alginate (ALG) (Pronova UPMVG, 60% guluronate, 40% mannuronate, M_{w}=120 k and 280 k) was purchased from Novamatrix, Norway. Hyaluronic acid (HA, M_{w}=200 k), poly(allylamine hydrochloride) (PAH, M_{w}=60 k), poly-L-lysine (PLL, Mw=50 k to 70 k), low molecular weight chitosan (LMWC, M_{w}=15 k), diethylaminoethyl dextran (DEAED, M_{w}=500 k) and all other reagents were purchased from Sigma Aldrich, USA. The anionic polymer and cationic polymers used are shown in FIG. 5 (a). FIG. 5 (a) depicts the name of molecular structures of anionic polymers and cationic polymers used.

Fabrication of Herringbone (HB) Microfluidic Chip

In some embodiments, negative photoresist (SU-8, MicroChem) was photolithographically patterned on silicon wafers to create masters with two-layer features. The first layer was the main microfluidic channel and the second layer forms the herringbone structures. The heights of SU-8 features ranged from 25 to 75 μm on the masters. Polydimethylsiloxane (PDMS, SYLGARD 184, Dow Corning) was poured, degassed, and cured in a conventional oven at 75° C. for 24 h. The cured PDMS replicas were removed from the molds, oxygen plasma treated, and bonded to glass substrates to form the final devices.

Biotin Modification of ALG and HA

In some implementations, LBL formation was modified for binding. Alginate and hyaluronic acid was modified with both N-(3-aminopropyl) methacrylamide hydrochloride (Polysciences 21200-5) and biotin hydrazide (Sigma B7639) using standard carbodiimide reaction.

Briefly, 1.0 wt % of ALG or HA solution was prepared in MES buffer, pH=6.0. Per 50 mL of ALG or HA solution, 80 mg of biotin hydrazide, 113 mg of methacrylamide, 360 mg of 1-ethyl-3-[3-dimethylaminopropyl]carbodiimide hydrochloride (EDC, Pierce 22980), and 204 mg of hydroxysulfosuccinimide (Sulfo-NHS, Pierce 24510) were added and reacted for 3 h, after which time the solution was dialyzed against dH₂O for 48 h and lyophilized. Alginate or hyaluronic acid was reconstituted at 2 mg/mL in dH₂O prior to use.

Preparation of LBL Films

According to various embodiments, LBL assembly of charged polymers were applied to build nanofilms inside microfluidic devices. The LBL layers formed from alternating bilayers of anionic and cationic polymers. The biotin modified ALG and HA were used to prepare anionic polymer solution, while PAH, PLL, LWMC and DEAED were used to prepare cationic polymer solutions. The initial experiments were performed using a simplified microfluidic device comprising a straight PDMS microchannel with the dimension of 400 μm (width)×100 μm (height)×10 mm (length) sealed on a glass substrate.

Briefly, glass substrate was treated with oxygen plasma for 5 minutes and immediately bonded to oxygen plasma treated PDMS replicas to form final devices. For LBL assembly of nano-films, a cationic polymer solution (2 mg/mL, pH 4.5) was first injected into the inlet of the device to occupy all the inside area, sit for 5 minutes for the absorption of polymers, then the solution was removed by air flow and the device was washed with 1 mL DI water for two times, then subsequent anionic polymer solution (2 mg/mL, pH 4.5) was injected into the device and allowed a 5 minutes absorption time, after which the device was washed with DI water. This process was repeated 5 times at room temperature under sterile conditions.

Degradation of LBL Films.

According to various embodiments, to visualize the degradation of LBL films, a solution of 0.05 mg/mL Streptavidin Dylight 650 fluorescent conjugates (Thermo Scientific) in PBS was introduced into the modified microchannels and stored at 4° C. for 4 hours. Then the avidin solutions were removed and the devices were washed thoroughly with DI water. The fluorescent intensity of each film was recorded using a fluorescent-optical microscope at the same exposure time of 2 seconds. A 2 mg/mL enzyme solution (alginate lyase or hyaluronate lyase in PBS containing 1 wt % bovine serum albumin, BSA) was introduced into the microchannel and kept flowing for 30 minutes at 2.5 mL/hr. After thoroughly washing the microchannel with DI water, the fluorescent intensity of the film was recoded using the same exposure time of 2 seconds. All the images were analyzed using ImageJ, and the fluorescent intensities of different types of films were normalized to the maximum intensity, which was obtained from ALG/LMWC film before degradation. The change of fluorescent intensity before and after degradation was calculated for all the film compositions.

Optimization of the Degradation of ALG/PAH LBL Film

In some embodiments, a series of ALG/PAH nano-films were made using the method described above. Four types of 2 mg/mL ALG solutions were prepared as following: ALG with molecular weight of 120 k at pH 4.5 and pH 7.2, as well as ALG with molecular weight of 280 k at pH 4.5 and 7.2. PAH used in this experiment was labeled with fluorescein isothiocyanate (FITC). For the comparison of degradation efficiency, fluorescent intensities of four films were recorded at t=0, 10 min, 20 min, and 30 min. Film thickness were measured using a profilometer. The morphology of films before and after degradation was determined using Atomic Force Microscopy.

Surface Modification of HB-Chips with ALG/PAH LBL Film

According to some embodiments, the surface of the inside wall of HB-chips was modified with ALG/PAH nano-films through LBL assembly as described above. Glass substrate was treated with oxygen plasma for 5 minutes and immediately bonded to oxygen plasma treated PDMS replicas to form final devices. PAH solution (2 mg/mL, pH 4.5) was first injected into the inlet of the device to occupy all the inside area, sit for 5 minutes for the absorption, then the solution was removed by air flow and the device was washed with 1 mL DI water for two times, then biotin modified ALG solution (2 mg/mL, pH 4.5) was injected into the device and allowed a 5 minutes absorption time, after which the device was washed with DI water. This process was repeated 5 times at room temperature under sterile conditions. A solution of 0.05 mg/mL Streptavidin in PBS was introduced into the device and stored in at 4° C. until use. Within 24 hr of the experiment, 20 μg/mL biotinylated goat antihuman EpCAM (R&D Systems) solution in PBS containing 1% BSA and 0.09% sodium azide were added to the devices. For coating IgG antibody, biotinylated normal goat IgG (R&D Systems) was in place of the biotinylated EpCAM. One hour prior to running the HB-Chip, devices were purged with 3% BSA with 0.05% Tween20 (Fisher Scientific) solution.

Cell Line Preparation

A human prostate cancer cell line, PC3 (ATCC, VA), were cultured at 37° C. in F-12K growth medium containing 1.5 mM L-glutamine supplemented with 10% FBS and 1% Penicillin/Streptomycin with media changes every two days. Cells were released from culture flasks through incubation in 0.05% Trysin—EDTA (Invitrogen, CA) at 37° C. for 5 minutes. Prior to spiking into whole blood, all cells were prelabeled with a fluorescent cellular dye following the manufacturers' protocol. The cell suspension was subsequently diluted to the desired concentration. Experiments were performed using PC3 cells suspended in serum-free medium or spiked into healthy donor whole blood.

Cell Capture and Release Experiment

Spiked cell experiments were performed with either the singe channel devices or HB-Chips. Experiments with the HB-Chip were processed with the standard CTC processing machine. Large HB-Chips were subsequently imaged and enumerated using automated image-processing system. Capture efficiency was calculated as the number of spiked cells captured in the HB-Chip divided by the total number of cells flowed through the device. For the releasing experiment, the device was first washed with PBS, then the alginate layse (2 mg/mL) in PBS containing 1% BSA was flowed through the device using a syringe pump (PHD 2200, Harvard Apparatus). The cell collection vial containing cell media was connected at the outlet of the device. Both cells remaining on the microfluidic device and in the collection vials were imaged and counted manually on a fluorescence microscope. Recovered cell viability was measured using a standard Live/Dead fluorescent assay (Life Technologies L3224) and compared to control cells that were never introduced into the microfluidic devices.

Study Subjects and Blood Processing

Patients with advanced prostate cancer were recruited according to a protocol approved by the institutional review board (IRB). Blood specimens from healthy volunteers were collected under a separate IRB-approved protocol. Cancer patients were treated at the Massachusetts General Hospital Cancer Center, donated 10-20 mL of blood on one or more occasions for analysis on the HB-Chip. All specimens were collected into vacutainer (Becton-Dickinson) tubes containing the anticoagulant EDTA and were processed through the HB-Chip within 6 hr of blood draw. Briefly, sample runs included a 5 mL aliquot of blood was placed in an air-tight conical tube on a rocker assembly, and ˜2 mL of blood were pneumatically driven through the chip at a flow rate of 1.5 mL/hr. Then, the HB-Chip was flushed with 2.5 mL of PBS at 2.5 mL/hr to remove any nonspecifically bound cells.

### Example 2

### Capture and Release of PC3 Cells from Microstructured Chips

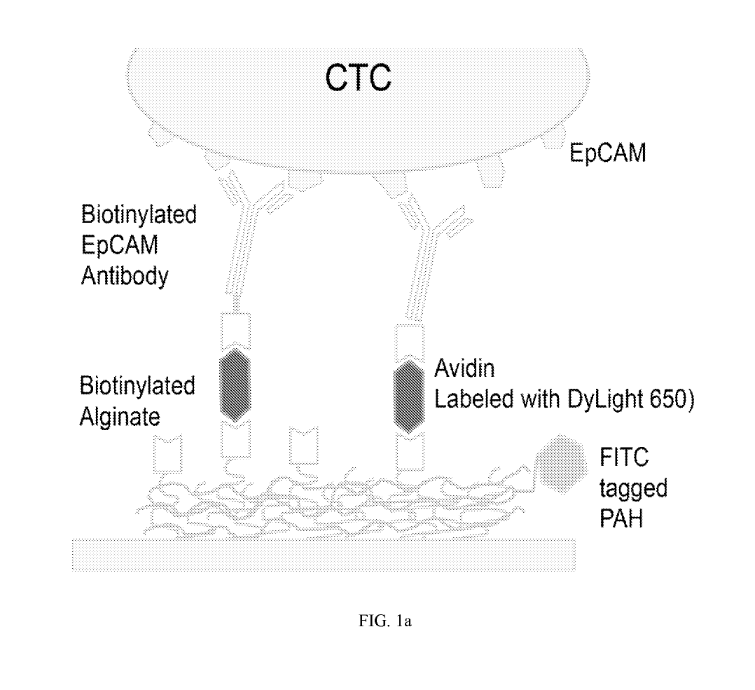

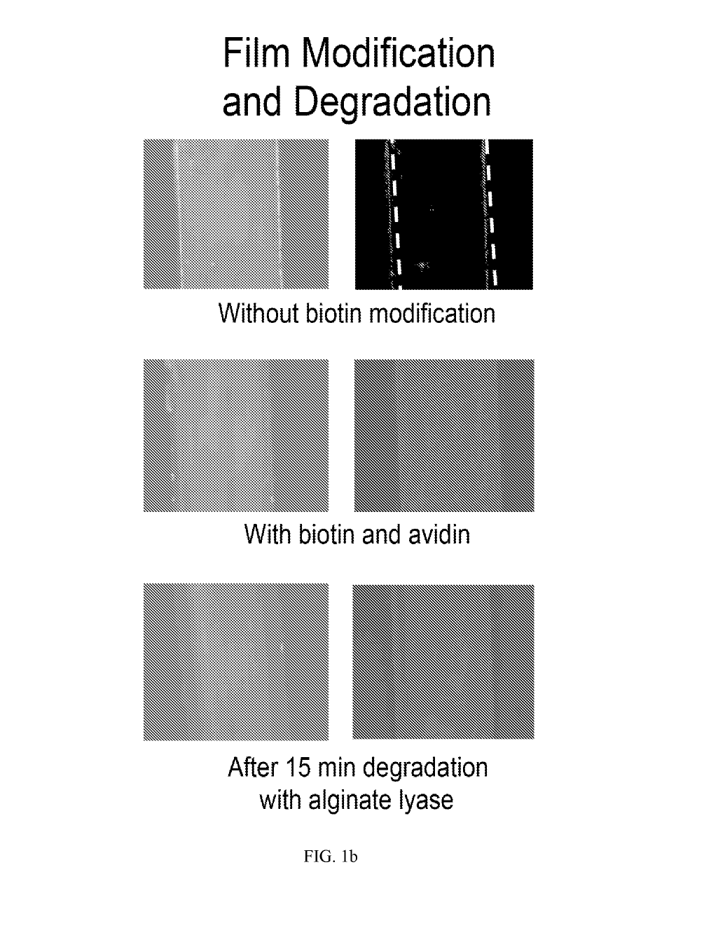

Films were constructed from alternating layers of biotinylated alginate and fluorescent poly(allylamine hydrochloride) (PAH) in a bilayer architecture within a PDMS microchannel on a plasma treated glass substrates using capillary force (manual pipetting, or liquid handling robots). Parameters such as polycation solutions, pH levels, deposition times, and number of bilayers can be used to make films with designed properties. The described films were then modified with dyed neutravidin, followed by modification with EpCAM antibodies. PC3 cells were cultured, and after being suspended in serum-free media, were flowed through the microfluidic channels in order to test how effectively the modified film could capture CTCs. Alginate lyase, an enzyme that cleaves the alginate backbone of the polyanion used to build the film, was then flowed through the channel in order to test the degradation of the films and the efficiency of cell release. FIG. 1(a) is a schematic of the architecture of the LBL film and the capture of cells.

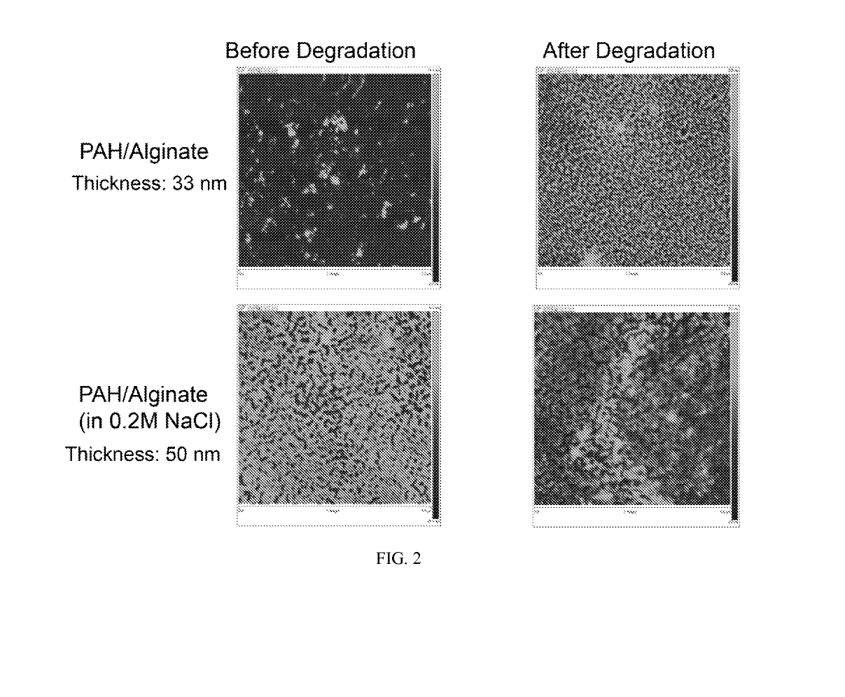

Tests were performed to optimize the multilayer films built within the microchannels. Fluorescence pictures were taken to confirm that the film building and modification with avidin. FIG. 1 (b) shows the degradation of the film after exposure to alginate lyase. The film thickness and roughness were measured both by profilometery as well as atomic force microscopy (AFM). Parameters such as the pH of solutions, number of bilayers, polycation, and salt concentration were varied. Experiments were performed with 0.2 M NaCl solutions to create a more porous film that could more easily degrade. The optimal pH of the polyelectrolytes and wash solution for the PC3 capturing example film described here was found to be 4.5, and the film was shown to increase in roughness after degradation. As shown in FIG. 2, AFM images demonstrate that the presence of salt effectively created nano-porous films.

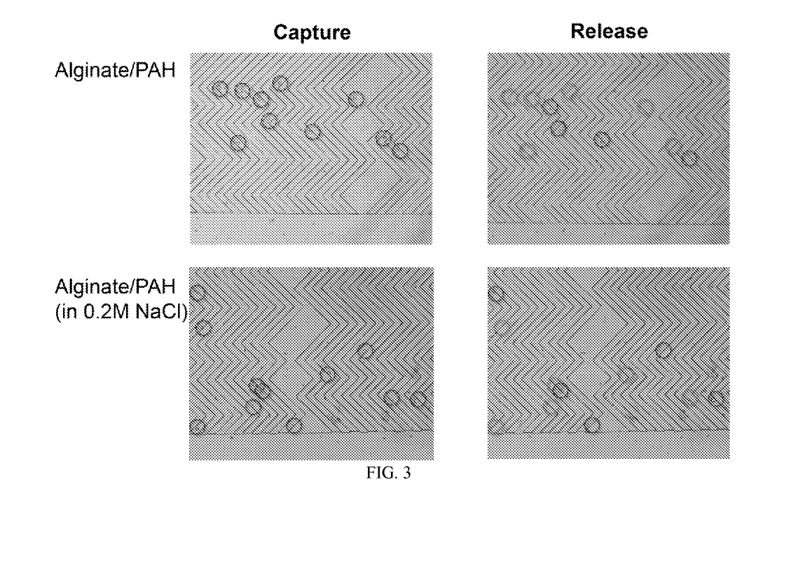

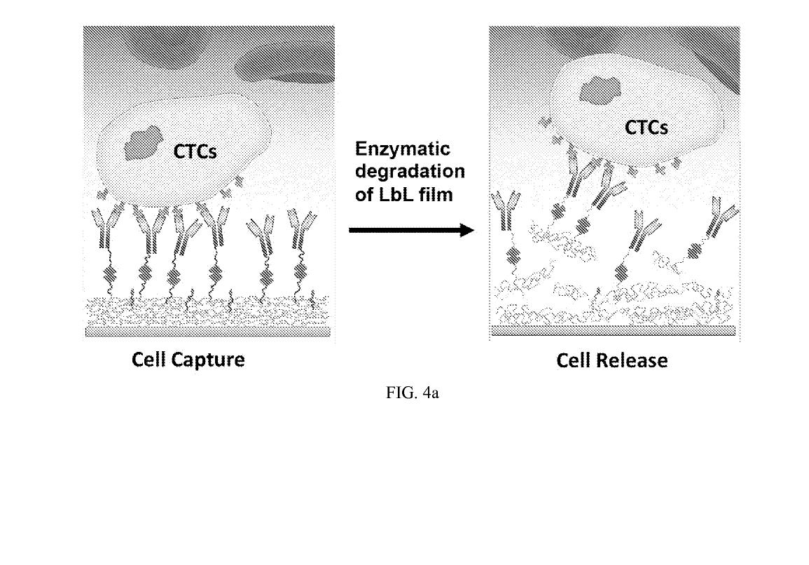

Various flow rates and times were tested for proper cell anchoring as well as degradation. Effective release was observed after flowing alginate lyase through the microchannels at 1-3 mL/hour for 30-60 minutes. FIG. 3 illustrates that cell experiments within the modified microstructured chips were able to demonstrate successful capture and release PC3 cells using PAH/alginate multilayer films degraded with alginate lyase.

Results and Discussion

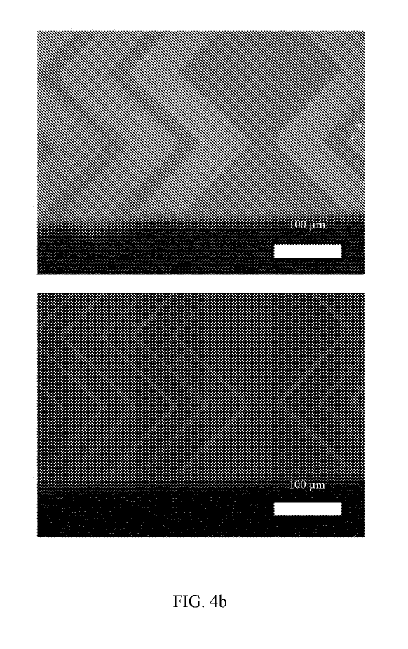

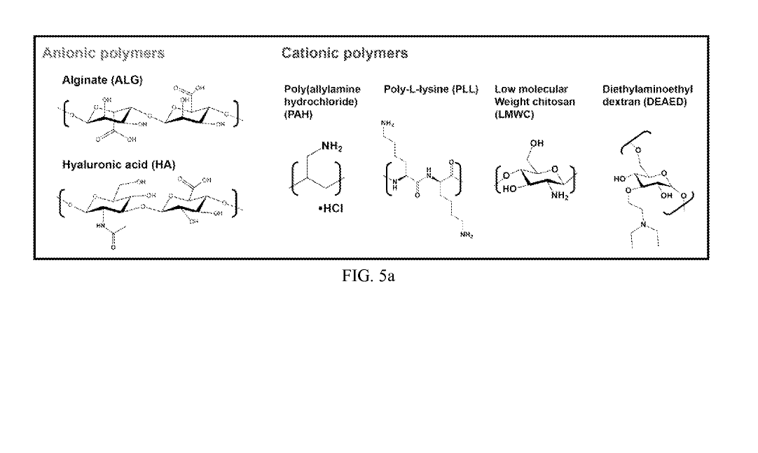

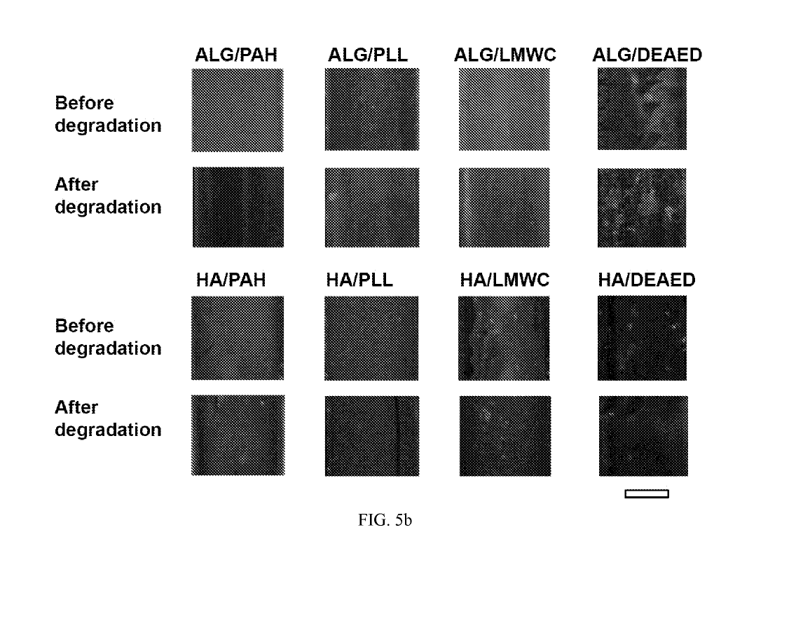

FIG. 4 (a) shows a schematic of capture of CTCs using microfluidic HB chips modified with enzymatically degradable LBL nano coatings. Through enzymatic degradation of the LBL film FIG. 4 (a) demonstrates cell release. Using a fluorescent image microscope, the top image of FIG. 4 (b) shows an image of the structure of the HB chip and the bottom image shows the surface of HB chip modified with of LBL film and the antibody linkages. As depicted LBL is the most efficient method to generate conformal coating of functional films inside microfluidic devices with complex microstructures.

LBL films of alginate (ALG) and Hyaluronic Acid (HA) before degradation and after degradation are illustrated in optical fluorescent images of FIG. 5 (b). Fluorescent Intensity (FI) for all films was measure and normalized by the intensity of ALG/LMWC before degradation. FIG. 5 (c) demonstrates fluorescent intensity (FI) change of the films before degradation are shown having bars with a black box, and the films after degradation are shown having a gray box. The white bars represent FI of series of films made by ALG and gray bars represent FI of series of films made by HA. The library of LBL films as shown in FIG. 5 is intended to be exemplary of the capabilities of the general structures and methods of the present invention.

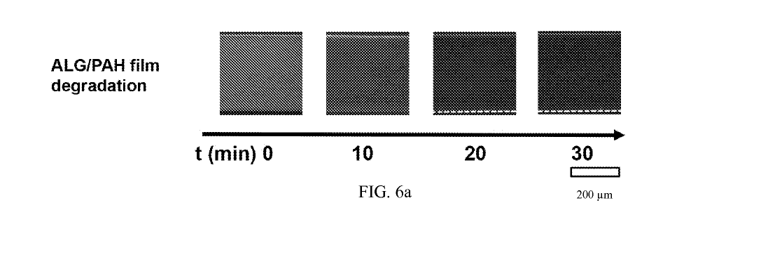

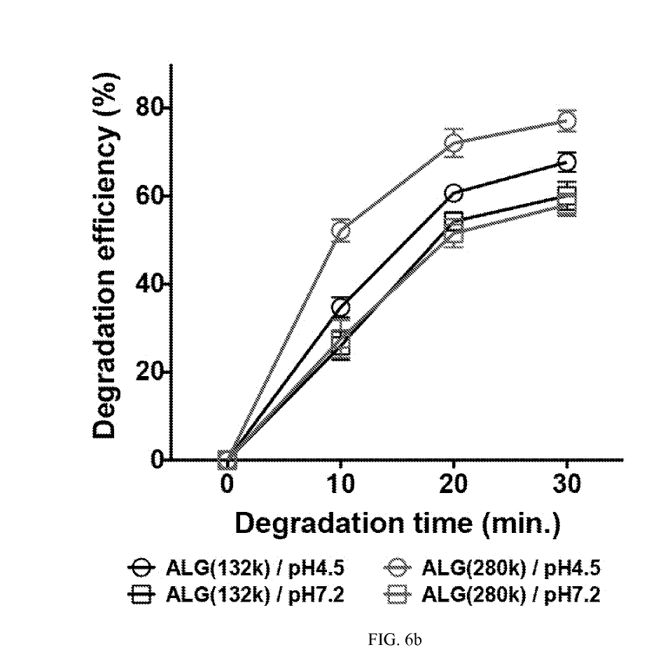

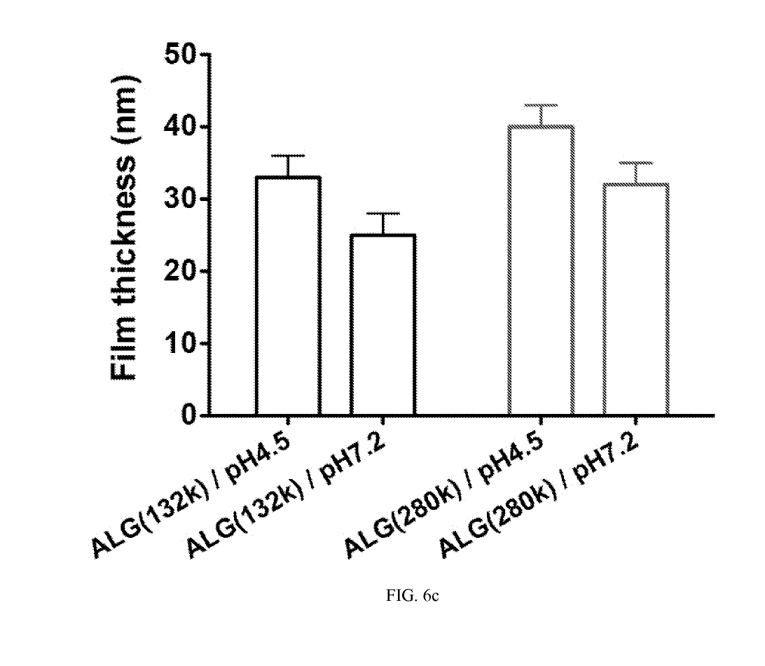

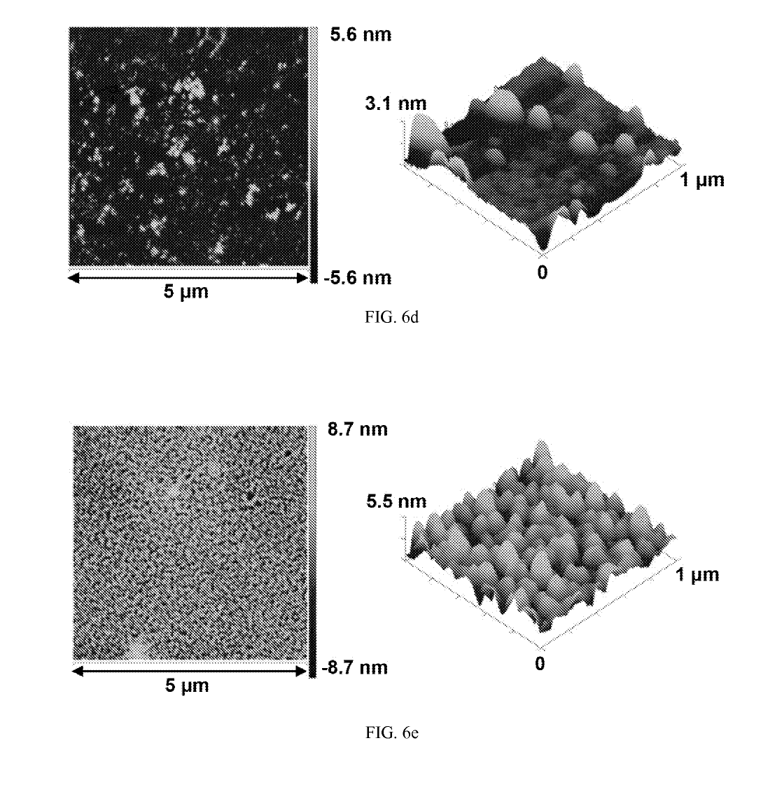

Capture and release of spiked PC3 cells from serum free media is shown in FIG. 6. The LBL film was made by FITC-labeled PAH and CY5-labeled avidin linker. The cells are stained with DAPI. Optical microscope images for PC3 cells when captured, when release, and after release are shown in FIG. 6 (a). Captured cells are indicated by blue circles, and released cells by red circles. Release efficiency at various degradation time and flow rates of enzyme solution is demonstrated in FIG. 6 (b). ALG film thickness for various compositions is shown in FIG. 6 (c). FIG. 6 (d) illustrates live/dead cell viability test of captured PC3 cells in the HB chip, thereby showing improved capability to prevent cell damage and death during capture and release. FIG. 6 (e) illustrates fluorescent microscope images of released PC3 cells that show small pieces of LBL films on the cell surface indicating successful cell capture and release without damaging the cell.

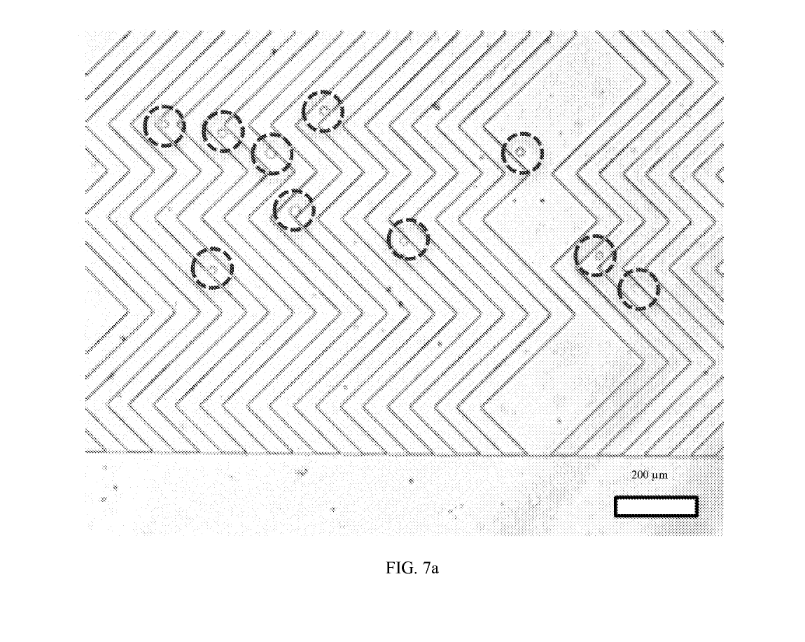

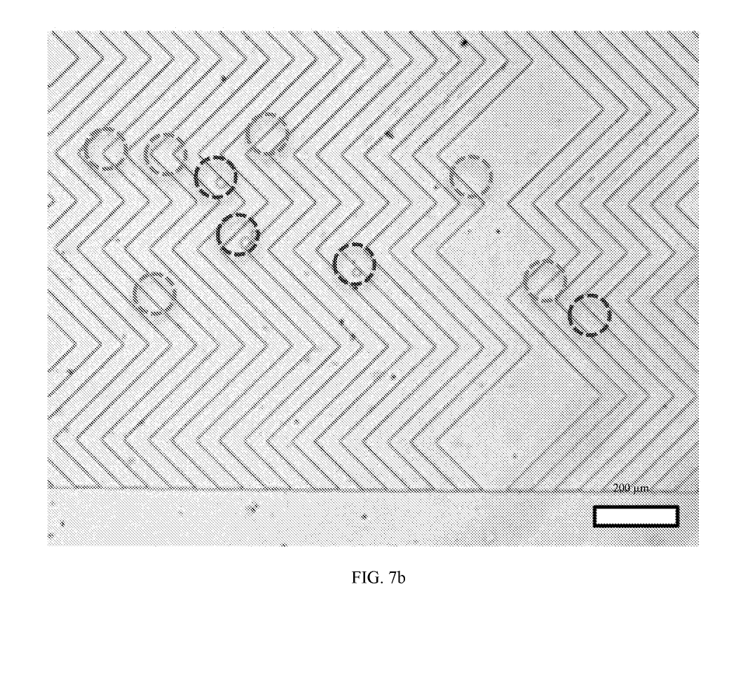

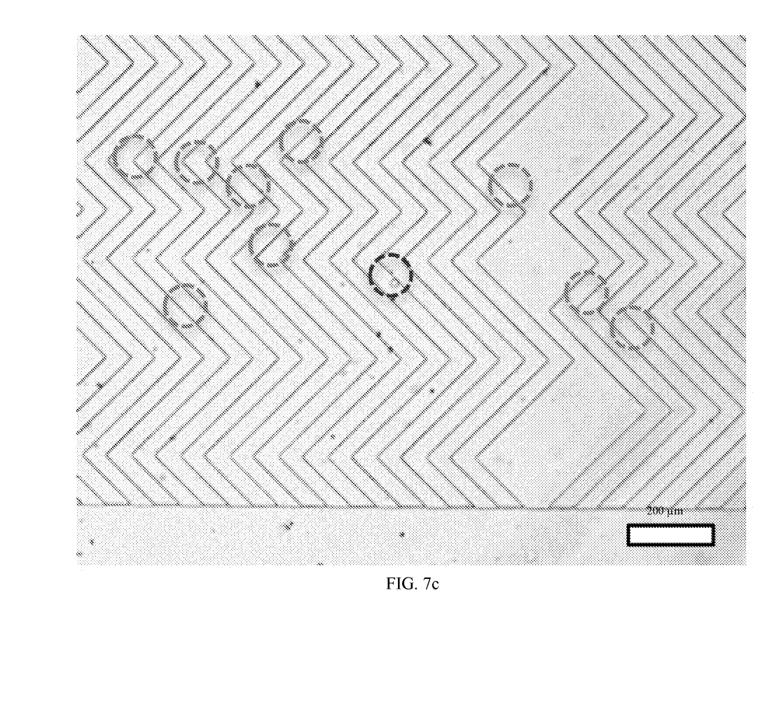

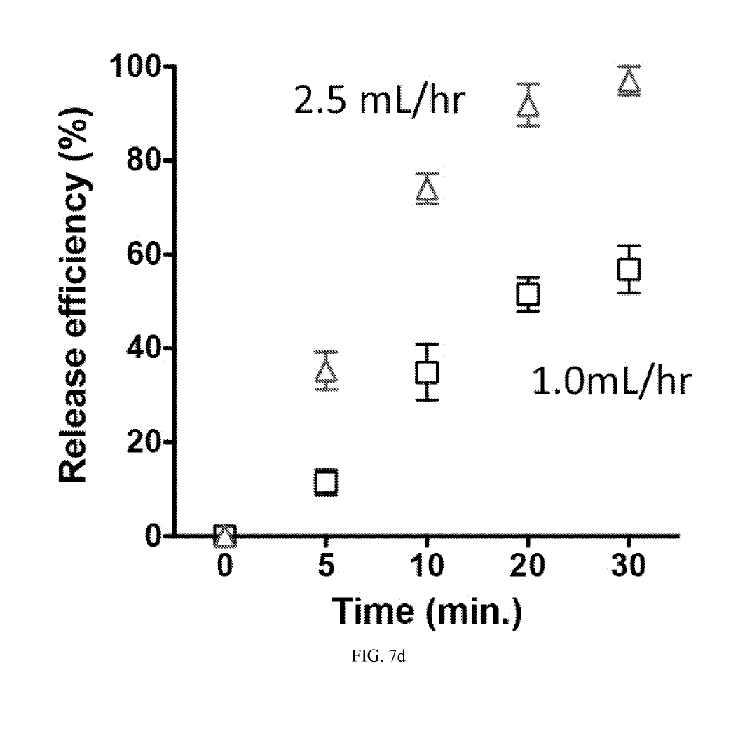

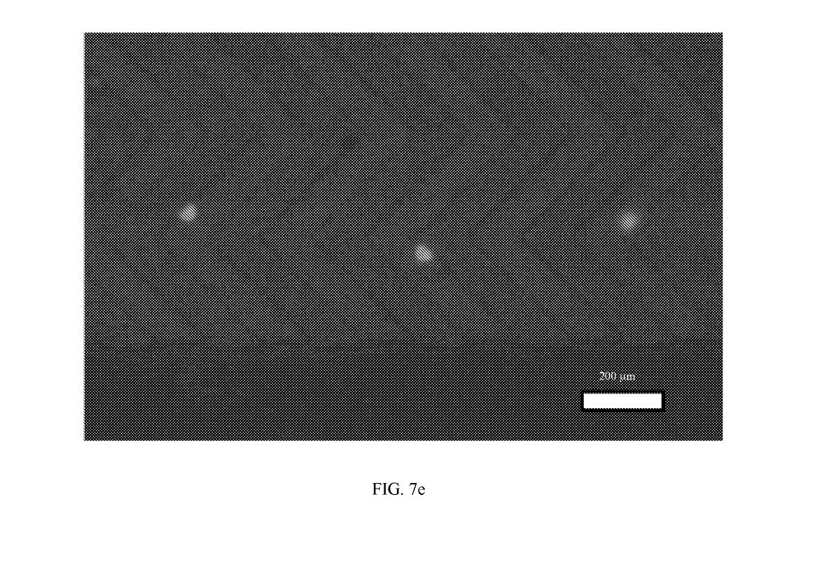

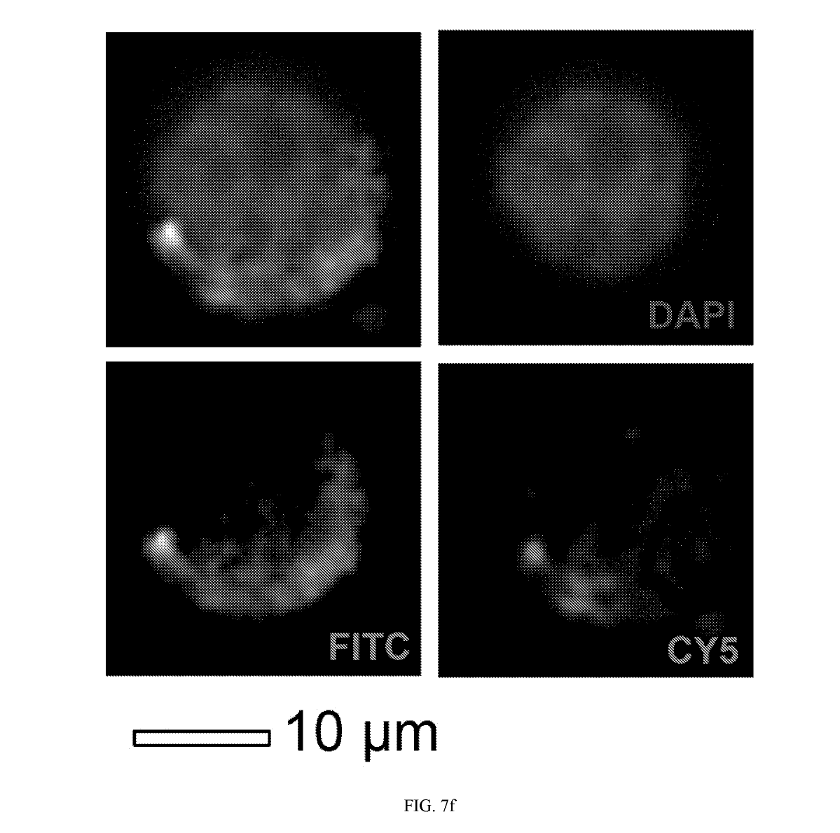

Release efficiency can be tuned by flow rate and concentration of enzyme solution. The LBL films were made by FITC-labeled PAH and CY5-labeled avidin linker. The cells are stained with DAPI. FIG. 7 demonstrates capture and release the spiked CTC cell lines, wherein cells were alive in the devices. Optical microscope images for PC3 cells when captured, when release, and after release are shown in FIGS. 7 (a)-(c). Release efficiency at various degradation time and flow rates of enzyme solution is depicted in FIG. 7 (d). Similarly, live/dead cell viability test of captured PC3 cells in the HB chip is shown in FIG. 7 (e) demonstrating improvement in preventing cell damage over prior techniques. Fluorescent microscope images of released PC3 cells of FIG. 7 (f) shows pieces of LBL films on the cell surface demonstrating successful capture and release.

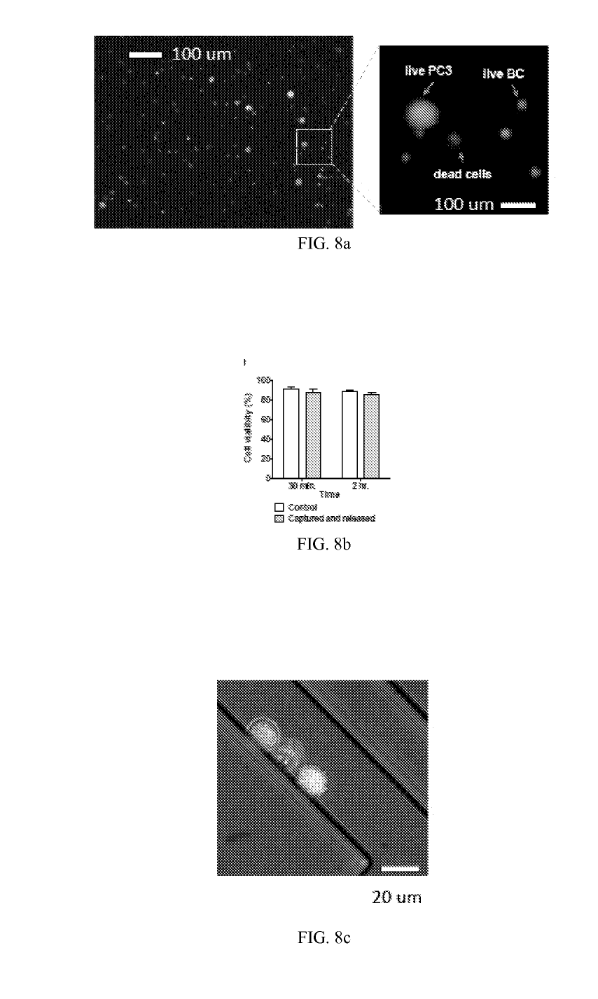

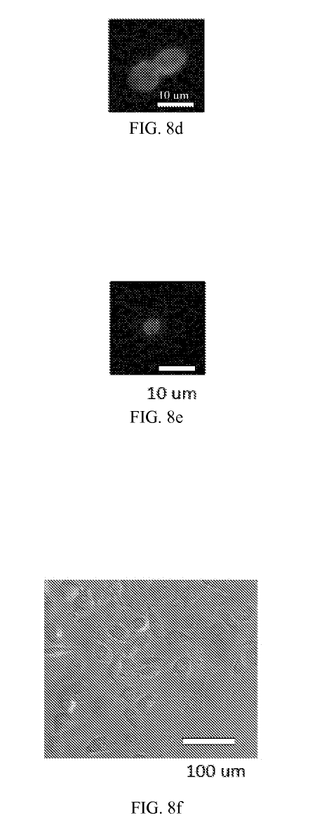

Capture and release of spiked CTC cell lines in whole blood has been achieved using the structures and methods of the present invention producing cells having good viability. Captured cells were confirmed to be PCs by EpCAM staining Released cells were cultured for 5 days and they can adhere to the surfaces of culture plate and proliferate, showing that the cells are not damaged from being captured and released. Released cells were evaluated for viability using a fluorescence. FIG. 8 (a) shows LIVE/DEAD assay. PC3 cells were pre-stained with cell tracker orange before spiking into blood. Live PC3 cells shown co-localization of orange color and green color. Cell viability of PC3 cells spiked in blood is shown in FIG. 8 (b). FIG. 8 (c) illustrates immunofluorescent staining of cell surface receptors for captured PC3 cells in the HB chip. FIG. 8 (d) shows released PC3 cells. FIG. 8 (e) shows white blood cells with EpCAM expression, DAPI nuclear staining, and CD45 expression. FIG. 8 (f) illustrates released PC3 cells showing proliferation and maintaining viability for culturing, image was taken after five days of releasing from HB chip.

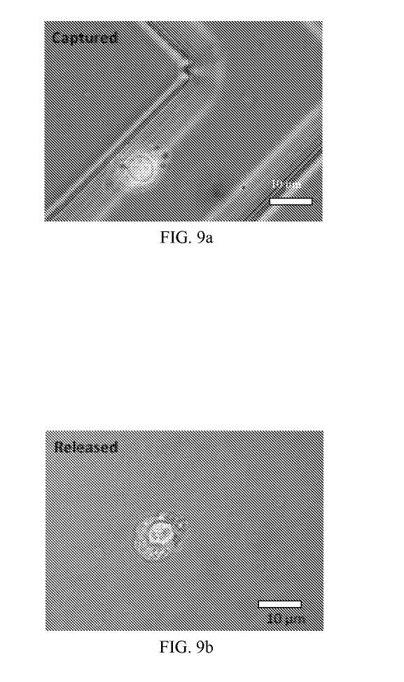

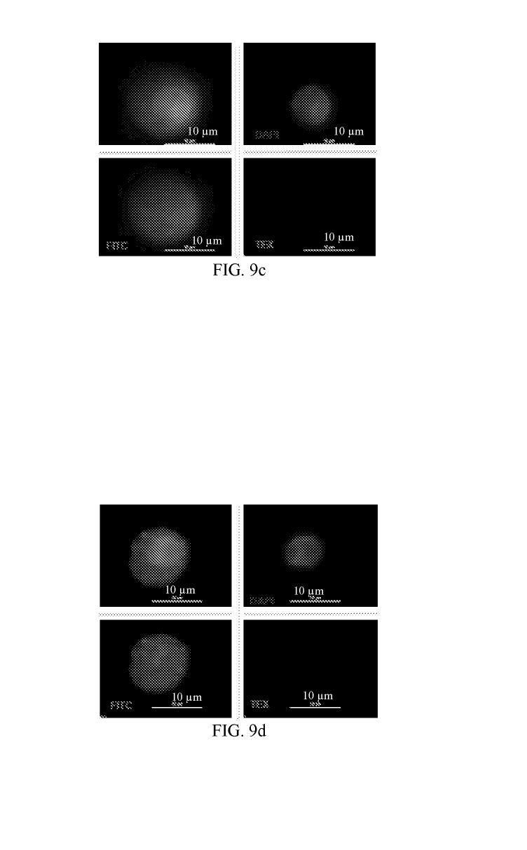

Capture and release CTCs from cancer patient bold sample is shown in FIG. 9. Captured cells are confirmed to be tumor cells by EpCam, HER2 and EGFR staining FIG. 9 illustrates immunofluorescent staining of cell surface receptors from the HB chip. FIG. 9 (a) shows captured CTC cells. FIG. 9 (b) illustrates released CTC cells from patient blood samples in the HB chip. FIGS. 9 (c) and (d) shown EpCAM, HER2 and MET expression in green, DAPI nuclear staining in blue, and CD45 expression in red.

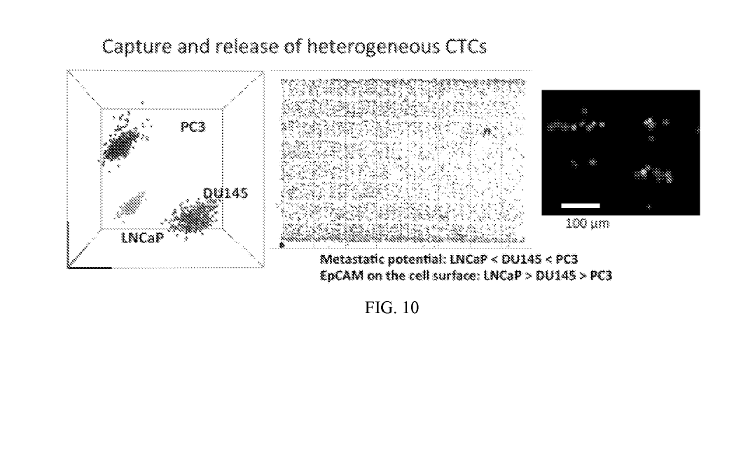

A mixture of three different human prostate cancer cell lines were captured on the same HB-Chip. The structures and methods of the present invention shown selective release these cells is possible. FIG. 10 shows the capture and release profiles for these heterogeneous CTCs.

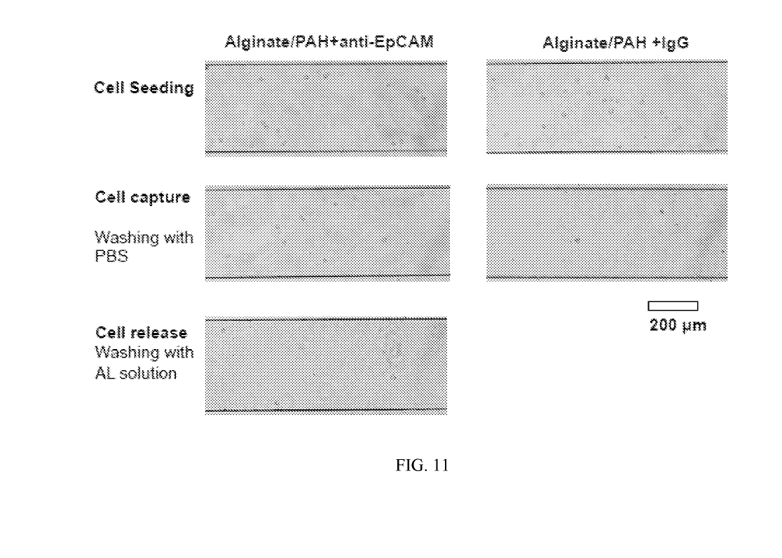

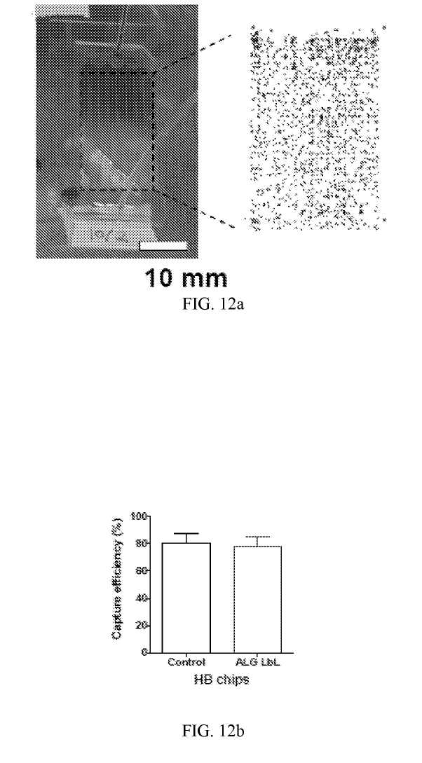

Spiked PC3 cells in whole blood were capture. FIG. 11 illustrates PC3 cell capture and release in single microfluidic device. FIG. 12 demonstrates the PC3 cells capture in HB chips. Blue bots represents the captured PC3 cells on HB-chips, showing about 80% of capture efficiency, which is consistent with PC3 cells suspended in serum-free medium.

It is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems of the present invention that consist essentially of, or consist of, the recited components, and that there are processes and methods according to the present invention that consist essentially of, or consist of, the recited processing steps.

Similarly, where articles, devices, and compositions are described as having, including, or comprising specific compounds and/or materials, it is contemplated that, additionally, there are articles, devices, mixtures, and compositions of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for organizational purposes and are not meant to be limiting.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A device, comprising:
a substrate having a surface; and
a layer-by-layer (LbL) film disposed on said surface, the LbL film comprising at least one multilayer unit including at least a first layer and a second layer non-covalently associated with one another,
wherein at least one of the first layer or the second layer of the at least one multilayer unit is decomposable, and at least one of the first layer or the second layer of the at least one multilayer unit comprises a binding agent that binds to an entity so as to retain the entity in association with the device.

2. The device of claim 1, wherein the first layer and the second layer of the at least one multilayer unit are non-covalently associated with each other by an interaction selected from: affinity interactions, metal coordination, physical adsorption, host-guest interactions, hydrophobic interactions, pi stacking interactions, hydrogen bonding interactions, van der Waals interactions, magnetic interactions, electrostatic interactions, and dipole-dipole interactions.

3. The device of claim 1, wherein at least one of the first layer or the second layer of the at least one multilayer unit is photodegradable.

4. The device of claim 1, wherein at least one of the first layer or the second layer of the at least one multilayer unit is biodegradable.

5. The device of claim 1, wherein the LbL film coats the entire surface of a substrate.

6. The device of claim 1, wherein the binding agent binds to an entity selected from the group consisting of: cells, small molecules, viruses, and nucleic acids.

7. The device of claim 6, wherein the cells are circulating tumor cells (CTCs).

8. The device of claim 7, wherein the CTCs are human prostate cancer (PC3) cells.

9. The device of claim 1, wherein at least one of the first layer or the second layer of the at least one multilayer unit comprises a polyelectrolyte.

10. The device of claim 9, wherein the first layer comprises a first polyelectrolyte having a first charge, and the second layer comprises a second polyelectrolyte having a second charge, the second charge being opposite to the first change, and the first layer and the second layer are disposed next to and associated with one another by charge-charge interactions.

11. The device of claim 1, wherein at least one of the first layer or the second layer of the at least one multilayer unit comprises an anionic polyelectrolyte comprising a functional group selected from the group consisting of carboxylate, sulfonate, sulphate, phosphate, and nitrate.

12. The device of claim 1, wherein at least one of the first layer or the second layer of the at least one multilayer unit comprises a cationic polyelectrolyte selected from the group consisting of poly(L-lactide-co-L-lysine), poly(serine ester), poly(4-hydroxy-L-proline ester), poly[α-(4-aminobutyl)-L-glycolic acid], and poly([β-amino ester)s, each of which is optionally substituted with a functional group selected from protonated amine and phosphonium.

13. The device of claim 12, wherein the first layer of the at least one multilayer unit comprises an anionic polyelectrolyte comprising a functional group selected from the group consisting of carboxylate, sulfonate, sulphate, phosphate, and nitrate, and the second layer of the at least one multilayer unit comprises a cationic polyelectrolyte selected from the group consisting of a, poly(L-lactide-co-L-lysine), poly(serine ester), poly(4-hydroxy-L-proline ester), poly[α-(4-aminobutyl)-L-glycolic acid], and poly([β-amino ester)s, each of which is optionally substituted with a functional group selected from protonated amine and phosphonium.

14. The device of claim 1, wherein the first layer of the at least one multilayer unit comprises alginate.

15. The device of claim 1, wherein the second layer of the at least one multilayer unit comprises polyallylamine hydrochloride (PAH).

16. The device of claim 1, wherein the first layer of the at least one multilayer unit comprises alginate and the second layer of the at least one multilayer unit comprises polyallylamine hydrochloride (PAH).

17. The device of claim 1, wherein the binding agent is a protein.

18. The device of claim 1, wherein the binding agent is biotin.

19. The device of claim 1, wherein the binding agent is avidin.

20. The device of claim 1, wherein the binding agent is an antibody or a functional fragment thereof.

21. The device of claim 20, wherein the antibody binds to the cell surface antigen EpCAM.

22. The device of claim 1, wherein the binding agent is a complex comprising at least two discrete entities that are bound to each other via a non-covalent interaction.

23. The device of claim 1, further comprising a detection moiety associated with at least one of the first layer or the second layer of the at least one multilayer unit.

24. The device of claim 1, further comprising a detection moiety associated with the binding agent.

25. The device of claim 1, wherein the binding agent interacts with at least one cell surface marker.

26. The device of claim 25, wherein the cell surface marker is selected the group consisting of surface proteins, glycoprotein, group of proteins, carbohydrates, antigens, immunoglobulin, and receptors.

27. The device of claim 1, wherein the at least one multilayer unit is a bilayer.

28. The device of claim 27, wherein the LbL comprises 3, 5, 10, 20, 30, 40, 50, 60, 70, 80, 90, 100, 150, 200, 300, 400, or 500 bilayer units.

29. The device of claim 1, wherein the at least one multilayer unit is a bilayer, the first layer of the at least one multilayer unit comprises alginate, and the second layer of the at least one multilayer unit comprises a detection moiety.

30. The device of claim 1, wherein the at least one multilayer unit is a tetralayer.

31. The device of claim 1, wherein at least one of the first layer or the second layer is the same in each multilayer unit.

32. The device of claim 1, wherein the device further comprises at least one payload entity.

33. The device of claim 1, wherein the substrate is a microfluidic chip.

34. A method of capturing a target entity, comprising steps of:
providing a device comprising:
a substrate having a surface; and
a layer-by-layer (LbL) film disposed on said surface, the LbL film comprising at least a first layer and a second layer non-covalently associated with one another,
wherein at least one of the first layer or the second layer of the at least one multilayer unit is decomposable, and at least one of the first layer or the second layer of the at least one multilayer unit comprises a binding agent that binds to the target entity so as to retain the target entity in association with the device; and
exposing the device to a sample comprising the target entity so that the target entity is retained in or on the device.

35. The method of claim 34, wherein the step of exposing comprises exposing the device to a sample of body fluid.

36. The method of claim 35, wherein the sample of body fluid is selected from the group consisting of: serum, plasma, lymph fluid, synovial fluid, follicular fluid, seminal fluid, amniotic fluid, mild, whole blood, sweat, urine, cerebrospinal fluid, saliva, semen, sputum, tears, perspiration, mucus, tissue culture medium, tissue extracts, and cellular extracts.

37. The method of claim 34, further comprising a step of degrading at least one of the first layer or the second layer of the at least one multilayer unit, thereby releasing the target entity retained in or on that layer.

38. The method of claim 37, wherein the step of degrading includes contacting the device with a hydrolytic agent, thereby releasing the target entity.

39. The method of claim 37, wherein the step of degrading includes contacting the device with an enzymatic agent, thereby releasing the target entity.

40. The method of claim 37, further comprising a step of analyzing the target entity that is released.
